(19) United States
(12) Patent Application Publication
(10) Pub. No.: US 2026/0234567 A1
(43) Pub. Date: Aug. 13, 2026

(54) FLUORESCENT CONE REPORTER IPS CELLS, RETINAL ORGANOIDS AND USES THEREOF

(71) Applicant: The Saban Research Institute; Children's Hospital Los Angeles, Los Angeles, CA (US)

(72) Inventors: David Cobrinik, Los Angeles, CA (US); Jinlun Bai, Los Angeles, CA (US)

(21) Appl. No.: 19/160,744

(22) PCT Filed: Feb. 28, 2024
(86) PCT No.: PCT/US2024/017754

§ 371 Date: Aug. 28, 2025

Related U.S. Application Data
(60) Provisional application No. 63/448,844, filed on Feb. 28, 2023.

(51) Int. Cl. C12N 5/071; C12N 5/074; C12N 5/0793; C12N 9/22; C12N 15/11; C12N 15/85; G01N 33/50

(52) U.S. Cl. CPC C12N 5/0697; C12N 5/062; C12N 5/0696; C12N 9/22; C12N 15/111; C12N 15/85; G01N 33/5058; C12N 2310/20; C12N 2503/02; C12N 2503/04; C12N 2506/45; C12N 2510/00

(57) ABSTRACT

Fluorescent reporter pluripotent stem cell (PSC) lines are powerful tools to investigate cell type-specific development and disease phenotypes. When combined with live imaging, they enable direct and repeated observation of cell behaviors within a developing tissue. Provided herein is a novel human cone photoreceptor reporter line and retinal organoids that can track cone development.

### PRIORITY

This application claims the benefit of priority to U.S. Provisional Patent Application Ser. No. 63/448,844, filed Feb. 28, 2023, the complete disclosures of which is incorporated herein by reference in its entirety.

### GOVERNMENT FUNDING

This invention was made with government support under CA137124 awarded by the National Institutes of Health. The government has certain rights in the invention.

### BACKGROUND

Cone photoreceptors are needed for color vision and are impaired in retinal diseases such as cone-rod dystrophy, retinitis pigmentosa, Leber congenital amaurosis and retinoblastoma (Mustafi et al., 2009; Xu et al., 2014). Modeling of human cone development and disease in animals is challenged by human-specific cone development features (Singh et al., 2018).

### SUMMARY

Fluorescent reporter pluripotent stem cell (PSC) derived retinal organoids are powerful tools to investigate cell type-specific development and disease phenotypes. When combined with live imaging, they enable direct and repeated observation of cell behaviors within a developing retinal tissue. Provided herein is a human cone photoreceptor reporter line generated by CRISPR/Cas9 genome editing of WTC11-mTagRFPT-LMNB1 human induced pluripotent stem cells (iPSCs) by inserting enhanced green fluorescent protein (EGFP) coding sequences and a 2A self-cleaving peptide at the N-terminus of Guanine Nucleotide-Binding Protein Subunit Alpha Transducin 2 (GNAT2). In retinal organoids generated from these iPSCs, the GNAT2-EGFP allele robustly and exclusively labeled both immature and mature cones starting at culture day 34. Episodic confocal live imaging of hydrogel immobilized retinal organoids allowed tracking of morphological maturation of individual cones for >18 weeks and revealed inner segment accumulation of mitochondria and growth at 12.2 cubic microns per day from day 126 to day 153. Immobilized GNAT2-EGFP cone reporter organoids provide a valuable tool for investigating human cone development and disease.

Provided herein are methods for making in vitro retinal cultures, tissue, or retinal organoids, from pluripotent cells as well as the retinal tissue and retinal organoids themselves, in particular cell type specific (cone specific) reporter, e.g., EGFP. Retinal organoids replicate many characteristics of the retina (e.g., human or mammalian), and methods of using this retinal organoid to study disease and to identify therapeutic agents for the treatment of retinal diseases and disorders are provided, in particular with a cone specific reporter (e.g., EGFP).

One aspect provides a pluripotent stem cell line with a genetic modification that allows expression of either a) a marker protein and/or or b) a tamoxifen inducible Cre recombinase under control of the endogenous GNAT2 promoter.

One aspect provides a pluripotent stem cell derived, in vitro generated, retinal cell line, retinal tissue or retinal organoid (ROs) comprising either a) a marker protein and/or or b) a tamoxifen inducible Cre recombinase under control of the endogenous GNAT2 promoter.

Another aspect provides a retinal organoid comprising a population of human pluripotent derived photoreceptor (PR) cells comprising either a) a marker protein and/or or b) a tamoxifen induced Cre recombinase under control of the endogenous GNAT2 promoter.

In one aspect, the pluripotent stem cell is a human embryonic stem cell (hESC) or a human induced pluripotent stem cell (hiPSC). In another aspect, the marker is a fluorescent protein. For one aspect, the fluorescent protein is enhanced green fluorescent protein (EGFP).

One aspect provides a method of producing a retinal organoid, wherein an initial mass (similar to an embryoid body) is created with the genetically modified cells described herein and allow differentiation into forebrain-like cells, then retinal vesicle-like mass forms, followed by an optic cup like mass, then a retinal organoid.

In aspect, the marker protein is expressed in immature and mature cone cells in retinal tissue and/or retinal organoids. In another aspect, there is minimal disruption of normal cone development.

One aspect provides a method for obtaining a retinal tissue or a retinal organoid (RO) comprising culturing the modified pluripotent cell line described herein under conditions which allow the cells to differentiate into retinal cells and then form retinal tissue and/or retinal organoid. In one aspect, the ROs are embedded in a hydrogel. In one aspect, the hydrogel comprises hyaluronic acid (HA) and gelatin. In one aspect, the HA is thiolated-HA. In one aspect, the hydrogel comprises thiol-modified hyaluronan, a thiol-reactive crosslinker (e.g., polyethylene glycol diacrylate), and thiol-modified denatured collagen. One aspect further comprises episodic live imaging and assessment of cone morphological changes, inner segment development and mitochondria localization. In aspect, the embedded ROs are embedded and optionally imaged for more than 6 weeks without impaired photoreceptor development.

One aspect provides a method of testing for an effective therapeutic treatment for a retinal disease/disorder comprising: a. producing a retinal organoid comprising the methods described herein, wherein the retinal organoid optionally has a genetic mutation or other impairment, b. administering a candidate therapeutic treatment to the retinal organoid of a); and c. determining the effect on the retinal organoid of b). In one aspect, the candidate therapeutic effect on the organoid is monitored overtime with episodic live imaging and optionally therapeutic is administered more than one time. In one aspect, the retinoblastoma 1 gene (RB1; human gene ENSG00000139687 (location: Chr 13: 48.3-48.6 Mb); human protein NP_000312 or NP_000312.2; human mRNA NM_000321) gene is knocked out. In one aspect, the candidate therapeutic treatment comprises a protein, a virus, a RNA molecule, a DNA molecule, a gene therapy, a small molecule, a gene editor, a base editor, an RNA editor, a small molecule targeting DNA/RNA, a cell therapy, a genome or base editing technology or a nanoparticle.

### DRAWINGS

FIGS. 1A to 1D. Generation of GNAT2-EGFP cone reporter iPSC line. (A) Strategy for EGFP-P2A knock-in at the GNAT2 N-terminus. EGFP-P2A cassette is inserted after the endogenous GNAT2 ATG start codon. The sgRNA spans knock-in junction. LHA, left homology arm; RHA, right homology arm. Blue arrowheads: location specific genotyping primers. Red arrowheads: insert flanking genotyping primers. (B) Schematic of RNA and protein expressed by wild type and GNAT2-EGFP alleles. After translation and P2A cleavage, 18 P2A amino acid residues are added to the C-terminus of EGFP while a proline residue is added to the N-terminus of GNAT2. (C) Homology donor template map with EGFP-P2A cassette flanked by GNAT2 LHA and RHA. (D) Genotyping PCR with insert specific primer pairs. The 1 kb band in C-6, C-9, C-36. C-37, and C-41 indicate knock in of the EGFP-P2A cassette with correct orientation.

FIGS. 2A to 2F. Generation and characterization of GNAT2-EGFP retinal organoids (ROs). (A) Overview of the RO differentiation protocol. D, day; MM, maintenance medium; RA, retinoic acid; RPE, retinal pigment epithelium. (B) Representative phase-contrast images of GNAT2-EGFP ROs at day (d) 6, d29, d69, d162 and d245, and fluorescent image at d260. White dotted lines in the d29 image indicate presumptive developing neural retina. Arrowhead in the d245 image indicates a visible brush border on a mature RO. Scale bars: 100 μm. (C,D) Representative immunostaining of d105 GNAT2-EGFP RO, indicating co-expression of EGFP and ARR3 (C) or EGFP and RXRγ (D). Scale bars: 20 μm. (E) Quantification of cells expressing EGFP only, ARR3 only or both in nine sections from four ROs, two independent differentiations. (F) Quantification of cells expressing EGFP only, RXRγ only or both in eight sections from four ROs, two independent differentiations.

FIGS. 3A to 3E. Live confocal imaging of GNAT2-EGFP ROs. (A) Representative 3D renderings of Z-stack live confocal images of GNAT2-EGFP ROs at d62, d83, d111, d147 and d195. EGFP+ cells adopted mature cone morphology in late time points (d147 and d195). Scale bar: 50 μm. (B) Single Z-section from the Z-stack images from (A) in which nuclei are marked by mTagRFPT-Lamin B1 and cones are marked by EGFP. Scale bar: 50 μm. (C) Representative 3D rendering of live confocal Z-stack images of d245 GNAT2-EGFP RO incubated with MitoView. Scale bar: 50 μm. (D) Cross-section images from the Z-stack image in (C) showing coalescence of mitochondria at the EGFP+ cone inner segments above the nuclear layer stained with SPY555 DNA. (E) Immunohistochemistry of d245 GNAT2-EGFP RO confirming coalescence of TOM20+ mitochondria with EGFP+ cone inner segments. Scale bar: 20 μm.

FIGS. 4A to 4I. Live confocal imaging of hydrogel embedded GNAT2-EGFP ROs. (A) Schematic of RO embedding and imaging in hydrogel on Millicell™ cell culture insert (created with BioRender.com). ROs are submerged under media and imaged through the bottom of the dish. (B) Phase-contrast image of a d203 RO embedded in hydrogel. Scale bar: 100 μm. (C) Phase-contrast image of a hydrogel embedded d203 RO showing visible photoreceptor inner segment protrusion on the RO surface. Scale bar: 100 μm. (D) Representative 3D renderings of Z-stack live confocal images of hydrogel embedded GNAT2-EGFP ROs at d154 (15 days in hydrogel), d217 (78 days in hydrogel) and d267 (128 days in hydrogel). White arrowhead indicates the same group of three cones at each imaging time point. Scale bar: 50 μm. (E) Representative 3D renderings of Z-stack live confocal images of hydrogel embedded GNAT2-EGFP ROs at d126, d139 and d153 (upper panel) and the EGFP based segmentation result (lower panel). EGFP+ cone inner segment development was tracked. Magenta arrowheads indicate the seven cones tracked and quantified for RO #1 inner segment growth. Scale bar: 50 μm. (F) Pooled quantification of the inner segment volumetric change from 24 cones on three ROs from d126 to d153. (G-I) Inner segment volumetric change of individual cones from d126 to d153.

FIGS. 5 to 10 are directed to modeling retinoblastoma using retinal organiods

FIGS. S1A to S1O. Cone-specific GNAT2 expression in scRNA-seq analyses. (A) Expression of seven cone markers in developing fetal retina. Bulk RNA-seq data from Hoshino et al., 2017. (B-D) 3D UMAP display of scRNA sequencing of early stage ROs, fetal retinae, and adult retinae from Lu et al., 2020, by cell types (B), tissue age (C) and GNAT2 expression (D). (E) Violin plot of GNAT2 expression by cell type from the same scRNA sequencing dataset. (F-L) 2D UMAP display of scRNA sequencing of FACS enriched fetal retinal progenitor cells (RPCs) and photoreceptors from Shayler et al. (submitted) by cell type (F) and by expression of cone markers GNAT2 (G), GNGT2 (H), THRB (I), RXRG (J), ARR3 (K), OPNISW (L), and OPNILW (M). (N) Violin plots of cone marker expression by cell type in the same full-length scRNA sequencing dataset. (O) Exon coverage plot of GNGT2 and GNAT2 reads by cell type in the same full-length scRNA sequencing. ENSEMBL transcript isoforms are shown below each plot. No cell type specific isoform usage was detected.

FIGS. 11A-11C. Conditional cone-specific gene regulation strategy. A. Biallelic GNAT2 editing. Left, HDR donor vectors for GNAT-ER^{T2}CreER^{T2} (top) and GNAT2-EGFP (bottom). Right, PCR genotyping showing monoallelic EGFP knock-in C-6 and C-37. B. HDR donor vectors for CAG-LSSL to control expression of a gene of interest (GOI), dCas9-mediated CRISPRi or dCas9-mediated CRISPRa, with T2A-mTurquoise2 (mT2) to monitor induction. CAG-mT2 was inserted into AAVS1 and showed fluorescence in pilot studies. Check marks: constructs with verified correct insertion into GNAT2-EGFP iPSCs. C. Structure of a flowed gene (or exon) of interest, which will be combined with GNAT2^{/Cre} cells.

FIGS. S2A-S2C. GNAT2-EGFP C-41 genotyping, off target sequencing, and karyotype. (A) Sanger sequencing of the 5′ end and 3′ knock-in junctions showing expected junction sequences. (B) Sanger sequencing showing no mutations detected at the top five predicted off target sites of the gRNA used for CRISPR knock-in. (C) GNAT2-EGFP C-41 shows normal karyotype.

### DETAILED DESCRIPTION OF THE INVENTION

hPSC-derived ROs with cell type-specific fluorescent reporters can be used to monitor a cell's normal and disease-related behaviors. Photoreceptor reporter lines have been generated by introducing a CRX-GFP cassette into the AAVS1 locus (Kaewkhaw et al., 2015) or by inserting a Crx-mCherry transgene (Gasparini et al., 2022), a rod reporter line was made by replacing the NRL coding sequence with EGFP (Phillips et al., 2018), and cone reporter lines were produced by inserting a mouse cone-arrestin (mCar)-GFP transgene (Gasparini et al., 2022) or inserting T2A-mCherry at the C terminus of GNGT2 (Nazlamova et al., 2022). Additional lines reporting one or more cell types include a retinal ganglion cell (RGC) reporter line produced by inserting a P2A-tdTomato-P2A-Thy 1.2 cassette at the C terminus of BRN3B (Sluch et al., 2017), a SIX6-GFP/POU4F2-tdTomato double reporter line that separately labels all retinal cells and RGCs, and a VSX2-Cerulean/BRN3b-EGFP/RCVRN-mCherry triple reporter line that differentially labels retinal progenitor cells, RGCs, and photoreceptors (Lam et al., 2020; Wahlin et al., 2021). These lines have been used to investigate retinal morphogenesis, improve organoid differentiation, and purify specific retinal cell types for transcriptome profiling and cell transplantation. Cell type-specific fluorescent reporter lines may also enable continuous live imaging to characterize developmental and disease processes, yet episodic long-term imaging of individual retinal cells has not previously been demonstrated.

To probe normal cone development and build a platform to study cone diseases, provided herein is the generation of a human cone reporter iPSC line in which EGFP is specifically expressed in both immature and mature cones with minimal disruption on normal cone development. GNAT2, which encodes the cone-specific cx-subunit of transducin, a G-protein that couples visual pigment opsin to the cone phototransduction cascade (Morris et al., 1997; Morris & Fong, 1993), was selected. Prior studies demonstrated that Gnat2^{-/-} mice exhibit complete loss of cone phototransduction without changes in rod phototransduction or in cone or rod morphology (Ronning et al., 2018). Human GNAT2 expression is limited to cones, controlled by a cone-specific promoter, and initially induced following the early cone lineage determinants RXRG and THRB but prior to mature cone markers ARR3, OPNISW or OPNILW (Hoshino et al., 2017; Morris et al., 1997; Welby et al., 2017) (data from (Hoshino et al., 2017)). However, unlike RXRG or THRB, GNAT2 does not downregulate in adult cones (Hoshino et al., 2017; Welby et al., 2017). In ROs, RNA-seq of CRX-GFP+ photoreceptors demonstrated a similar onset order, with GNAT2 first detected at day 37 (Kaewkhaw et al., 2015). These features suggested that the endogenous GNAT2 promoter linked to a fluorescent protein may serve as an ideal cone-specific reporter.

### Definitions

Various further features and aspects of the invention are defined in the claims.

Unless defined otherwise, all technical and scientific terms used herein have the meaning commonly understood by a person skilled in the art to which this invention belongs.

To assist the reader, the following terms have the meanings ascribed to them below, unless specified otherwise.

As used herein, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

Organoids are self-organizing, 3D cell cultures which are valuable in many applications such as; drug screening, toxicity, disease modelling, and regenerative medicine. The term “organoid” refers to an organized mass of cell types, generated in vitro that mimics at least to some degree the structure, marker expression, or function of a naturally occurring organ. Organoids can be derived from isolated primary progenitor cells or pluripotent stem cells which are directed towards differentiation pathways to yield the desired cell types.

The term “retinal organoid” refers to organoids which mimic human retinogenesis through formation of organized layered retinal structures that display markers for typical retinal cell types. Polypeptide or polynucleotide expression of cells within the organoid or the constituent tissues can be determined and/or compared by procedures well known in the art, such as western blotting, flow cytometry, immunocytochemistry, in situ hybridization, fluorescence in situ hybridization (FISH), ELISA, microarray analysis, RT-PCR, Northern blotting, or colorimetric assays, such as the Bradford Assay and Lowry Assay.

“Photoreceptor cells” or “PR cells,” as used herein, are a specialized type of neuroepithelial cell that is capable of visual phototransduction and comprises an inner-segment and an outer-segment. There are two types of PR cells: rods and cones. Rods are adapted for low-light vision, to view in grayscale, and cones are adapted for daylight vision, to view in color.

The term “pluripotent stem cells (PSCs),” also commonly known as PS cells, encompasses any cells that can differentiate into nearly all cells, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of totipotent cells, derived from embryos (including embryonic germ cells) or obtained through induction of a non-pluripotent cell, such as an adult somatic cell, by forcing the expression of certain genes.

The term “induced pluripotent stem cells (iPSCs),” also commonly abbreviated as IPS cells, refers to a type of pluripotent stem cells artificially derived from a normally non-pluripotent cell, such as an adult somatic cell, by inducing a “forced” expression of certain genes. iPS cell lines can include any iPS cell line, including but not limited to iPS-SB-Ad4; iPS-SBAd3; iPS-DF19-9; iPS-DF4-3; iPS-DF6-9; iPS (Foreskin); and iPS (IMR90).

The term “detect” refers to identifying the presence, absence or amount of an analyte to be detected.

The term “disease” refers to any condition or disorder that damages or interferes with the normal function of a cell, tissue, or organ. For example, retinal degenerative diseases such as Retinitis Pigmentosa and age-related macular degeneration (AMD) are the major causes of vision loss due to cell death or functional loss of photoreceptor cells (PRCs) and/or retinal pigment epithelium (RPE).

The term “marker” refers to any protein (such as fluorescent proteins) or polynucleotide analyte having an expression level or activity associated with a particular cell type.

The terms “treat,” treating,” “treatment,” and the like refer to reducing or ameliorating a disorder and/or symptom(s) associated therewith. It will be appreciated that, although not precluded, treating a disorder or condition does not require that the disorder, condition or symptoms associated therewith be completely eliminated.

As used herein, an “effective or sufficient amount” or “therapeutically effective amount” is an amount of an agent, such as a therapeutic agent, sufficient to evoke a specified cellular effect according to the present disclosure. For example, an effective or sufficient amount of a therapeutic agent effective for treating a genetic mutation is the amount of the therapeutic agent which results in a partial or full restoration of function of the gene when administered to a subject.

As used herein, a “therapeutic treatment” refers to treatment with a therapeutic agent to provide a positive clinical effect or therapeutic benefit, such as the restoration of function of a gene or a wild-type phenotype.

By “subject” or “patient” is meant a mammal, including, but not limited to, a human, such as a human patient, a non-human primate, or a non-human mammal, such as a bovine, equine, canine, ovine, or feline animal.

The term “agent” means any compound or substance such as, but not limited to, a small molecule, nucleic acid, polypeptide, peptide, drug, ion, etc. An “agent” can be any chemical, entity or moiety, including without limitation synthetic and naturally occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc.

The term “medium” (also referred to as a “culture medium” or “cell culture medium”) means a medium for culturing cells containing nutrients that maintain cell viability and support proliferation.

The term “expression” refers to the cellular processes involved in producing RNA and proteins and as appropriate, secreting proteins, including where applicable, but not limited to, transcription, translation, folding modification and processing. Expressed markers include RNA transcribed from a gene and polypeptides obtained by translation of mRNA transcribed from a gene.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual” (Sambrook, 1989).

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the embodiment(s) disclosed herein. The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” should be interpreted as “including but not limited to,” the term “having” should be interpreted as “having at least,” the term “includes” should be interpreted as “includes but is not limited to,”, the term “comprising/comprises” should be interpreted as “comprising/comprises but is not limited to” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Guanine nucleotide-binding protein G(t) subunit alpha-2 is a protein that in humans is encoded by the GNAT2 gene (Morris T A, et al. (October 1993). Genomics. 17 (2): 442-8. doi:10.1006/geno.1993.1345. PMID 8406495 and “Entrez Gene: GNAT2 guanine nucleotide binding protein (G protein), alpha transducing activity polypeptide 2”.). Human GNAT2 sequences: ENSG00000134183, mRNA NM_005272, NM_001377295 or NM_001379232; protein NP_005263, NP_001364224 or NP_001366161; Gene location: Chr 1:109.6-109.62 Mb). An example of Homo sapiens G protein subunit alpha transducin 2 (GNAT2), RefSeqGene on chromosome 1 (NCBI Reference Sequence: NG_009099.2) is provided:

| (SEQ ID NO: 33) | |
|---|---|
| 1 | cctttacaca gtgcctggca cgaagtgagg gcccagtgaa aggtagctgt tatcattgtc |
| 61 | ccaggattca ttttaagcag aagtcctgcc cctgcaatct cacttggtcg tctgttttct |
| 121 | tccggccggc gtaggtagga ggctgtgccc tgcaattctg gctccccttc ccacatttct |
| 181 | gtatttggtg gttaagagct ttaactctag ccagaatgtc tgggttcaaa tccagatttc |
| 241 | acctacttcc tgactgatac tgggcaagtt acctaacttc tctgtgcttc tatttcgtct |
| 301 | gtaaaatgag tgtaagagta cctgctttat atggtggtta agataaatga gttaatataa |
| 361 | gttaactgct tagaactgtg cctggcctgt agttagtact cagtattcat gtattaccat |
| 421 | tgctttagcc ttggccttgg ttctctattg acactcacag gttgattcac ttgcagccgt |
| 481 | tctggggtct atccaacaag catccccact gccaaggggt agataatccg acctaaaggg |
| 541 | aacttgtgga gggaagtccc taagacttca ctgaatataa ttaaatgcat tagcaggaat |
| 601 | taatctcttc cctattttcc ctcttgtggg aggcctttgc cccccacacc cagttgttct |
| 661 | aatgctgttt gtctggctcc tcccagttct taccactccc acagcagaga tccttcccaa |
| 721 | actctgcctc cctcttctct ccccacgctg gattctggac cctgaaccct tttctccacc |
| 781 | tgcatctggt gccacttcca ttgatccctg gctactgttc cctgaacaaa cttaaaaaaa |
| 841 | acttggcctc ttcacctcac acagaatcca taaaactaaa aagagaacag agataaccag |
| 901 | gtttacctaa tcaaaatagc aaagactaaa aaacaaaaaa ccaaaaaata aacgctaatg |
| 961 | caggttggca agggtatagt tagtgagatg ggagacacac acacacgtac atgtgcacaa |
| 1021 | acacattact ggtggccttg aacactgata taatctttct gaaagccatc tggtaatata |
| 1081 | gaatcaaaat tttcaaaaag gttcttatta tttgacccag taatcaactt tctggaagtt |
| 1141 | gtttttctaa aaagagctgc cagaaaaata tgtatgcaaa aagttgttca gcaccacaat |
| 1201 | gtgtattgca tagaaaagtt agatacttta aaggtcgaac aataggggaa tgtttatgta |
| 1261 | atattcatac catgaaatac agccattaaa aattgttctt ggagaaatgt ttaatgatgt |
| 1321 | ggaaatgttt acaatataaa gttaggtaaa agaatggatg caaaacacta tacacagtat |
| 1381 | ggtcctactt atgtgatata catgggtaga aaagaaacac accaacaagt ttaccaatga |
| 1441 | ttatctgtag tgatggaatt atgggttata tttattttct ttatatttat ttgcatttta |
| 1501 | gaaattataa tacataccca taacttttat aatcaagggg atagaaatca acaaaaatct |
| 1561 | gtaaaaatct gtaattgcct ccacagtccc cccaacccag ccttcccggg gtcttaaccc |
| 1621 | cagacttaaa ctattctgtt tacatttatg gcccactgaa ttggtcttgg ggcgcttagg |
| 1681 | gattttgagg ctgatggact ctcaagaatg aataaaatga atctcaaaaa tcaagcacca |
| 1741 | tgtaactaca taaatgtcat gtatcttctg ataagatgca aatgttgaca tagttttaaa |
| 1801 | gatagtatct tgcattgggc caggtctctt gagcatcctg cttaatgcag acctacctac |
| 1861 | ctcagcagcc aataggtggc aatccttgcc ctccacccag cccaggcaaa ccagttcatt |
| 1921 | agtcccttta tgctagtccc ctagtcccat ctgcaacaca cccttcactc tacaaccctc |
| 1981 | ccctatttcc aggtcatctg gtgtattgtt gctgctagga agtcggccct gattatagtt |
| 2041 | aggcatcaat ccctccaccc actccatacc agcaagaatc tcagtgaggt ttcattgtct |
| 2101 | acgcagaaca gagcttggga aataaaggag tgagtcatcc tatggcctgg gcttttttca |
| 2161 | cactgaggcc ttaacattca ttgcctttga gcctctgaga tattccctag ctccaaacac |
| 2221 | agcttcctcc cctcctagcc tgttccccac atggctcctt aactggccct cccggaatcc |
| 2281 | cagctcccta aagatggtgc actcctgttt ctctgaagaa gtgcttttct ttgcttctgg |
| 2341 | acttcagctc acttgtgatg tccgcctccc tagctaggcc agaacaatct tcacagatac |
| 2401 | taaacttggg agagaatatc tgcagctcca aaaaggtcct tggccaaggc aatttcccaa |
| 2461 | accatacctt atgcctaggc ccttctagaa acaagctgtt cagctctggt ttctcagctt |
| 2521 | ctcccagcag ccagctccct ctccccctca cctcagtcac ccagcattgt gtttatagca |
| 2581 | ccatacagct gtttccatct atacttgttt ataagacccc agtggatttt agtcaaaccc |
| 2641 | cttgccattg aaggcgcttc actggctggc tccaatttcc ctttcttgtc ttcatcttca |
| 2701 | cctcccaacc ctccactcca tgcacctttc actccagtcc agtcacatca aagtcccagt |
| 2761 | cttctctgag cacaccttgt actttgtgcc tctacctctt ttttcatgct ctttcctttc |
| 2821 | cccaaaatgc gtatctccca cttccactgt tattctgctt ctctgtggaa agtctattca |
| 2881 | tttcacaatc acccttttct ttcttccgct gtagaacttt gtcagtctga cagtcagatg |
| 2941 | tgtgtttgat tgcctttcct accaggggtg aatgctaggt cttaaacagc tctgaatttc |
| 3001 | ccagcactac tgcagtatcc ctttaactca tttgtttttc tgtgaatttc ccccacctct |
| 3061 | gcctatggac tggaaggtcc tgaaggtagt tcctcatcct tcatgtcctc ccactccatc |
| 3121 | tgggacaagt ctttacccac aattcactta agacaaggct taccacggat gtcagacaga |
| 3181 | tcccttaact caggaaacat gcccaaatca ctaagaaaca gtgatttact tttcattcaa |
| 3241 | aatgataatg gttataaagg gctgtgaatc aaaacacctg agctctggct tggcttctga |
| 3301 | taccaacttg cttattaatg tgaggatcac ttttctgtac ttcagttcac tcacctccaa |
| 3361 | aatggaacta gcactacata ccttatttac ttcatcagga aactgttcgt aaatgtgatc |
| 3421 | ataaagtggg acagccacag gaaaggcaca ggaactgacg atagaggcag aggcaggagg |
| 3481 | tcacagacat caggatgatg tatgcacagt aatggatctg tactctcagg ccaggcgagg |
| 3541 | gctagcagtt ccctctctca gcttagcagt gagaaagggg aacttccatc cagtaagtta |
| 3601 | gggaacaaag cagaagcagc acagaagttt gcagaggtag tagcagaaaa aggaacaagc |
| 3661 | ccactttccc tgtgtgtgaa acaacttagt ggttccttta acaagggctg ctgttataga |
| 3721 | gttcatgcat ttgtccatgc tttctctctc tggctactta tgtggaagta atagaaagaa |
| 3781 | ggatcaccgg tttgtctgtt tactaggcca tataactttg tttttttgtt tttgaaacag |
| 3841 | agtctcactc tgtcacccag gttggagtgc agtggcgcaa tctcggctca ctgcaacctc |
| 3901 | cacctcccgg gttcaagcga ttctcatgcc tctatctccc aagtagctgg gtgcatgcca |
| 3961 | ccatgctggg ctttttcttt tcttttcttt tctttttttt ttttttttct gagacggaat |
| 4021 | cttgctctgt tgcccaggct ggagtgcagt gcctcccggg ttcaagtgat tctcctgcct |
| 4081 | cagcctccca agtagctggg attacaggtg cctgccacca tgcctggcta atttttgtat |
| 4141 | tttttttaag tagagacagg gtttcaccat gttgaccagg ctgtctcgaa ctcctgacct |
| 4201 | cagatgatct gcccattttg gcctcccaaa gtgttggaat tacaggcgtg agccaccatg |
| 4261 | cccggcctaa tttttgtatt tttagtggat acgggatttc accatgttgg ccaggaactc |
| 4321 | ctgacctcag gtcatccacc cacctcagcc tcccaaagtg ctgggattac aggcatgcgc |
| 4381 | cactatgccc agctgacttt aaatgagtta cttaactctc aagtctcagt taatctagtc |
| 4441 | tgtaaattgg ggacaatagc agttattgtg ataattaact ggtatgagcc acttacattg |
| 4501 | ttcagcagtt attaattata ataactataa cataataatt attaatagtt ccaggcagcc |
| 4561 | ccttgaacag tgtagccttc agcatggctc tggcctcagt gtgagttacc agtattgtga |
| 4621 | gtacgctgga gcagggctag cagtggggta taattactga aaccttataa ccaagattcc |
| 4681 | aagatagtca caatttcaaa gagactctcc tgtttttcct taagtcaata aggacttgcc |
| 4741 | aatttgattg ggaaaacatg gctaccatgg aaagcacctc agcagctgca tatttctcca |
| 4801 | tcagccccta cagtcactga aaggtggtgc gaaggaggaa gataattagc tatggctcag |
| 4861 | ggtacctgat aggcggggag acctagattc tactcctgac cttcccagtc ccaactggcc |
| 4921 | actgctgcac aagtccagct ctaaaatgaa agagcaaatt acatccttcg acctcagtag |
| 4981 | ttctccacct tggctgtaca ctagaatcag ctggggacgt ttttaaatcc ttgtgcctgg |
| 5041 | actgcactcc agaccaatta aatcagtatt ttttaaaaag ctctccaggt aaatgccact |
| 5101 | gtacactaaa agttgagaac tgtttcaggc cagggttttt caaacttcac agatgtgata |
| 5161 | actacctgta ttagcttaaa atattttctc tggctctgga gtttgatgct aacataccat |
| 5221 | ttaaaaacat acactatttt aacatctcgg aaagcagggt aatcttctag tcagtggcac |
| 5281 | ctaagacttg aggaaacaca gacaccagca acccaggtga ttcttaggat taggtaagtt |
| 5341 | tagcggaaaa caggtcccta gggcagtact tcccaatctt taatgtcttt agagtcacct |
| 5401 | gagatcttgt taaactgcgg attctgaatc agaagttctg gattaaagcc tgagattctg |
| 5461 | catgtctaat gagcttccgg gtgatctgat gttgctggct cttgatccac attttgggca |
| 5521 | acaaaattct aaaacatctc cacctgagga ggctccgcca gcaacactca tttaagtaaa |
| 5581 | ctgaaataat tggcatagag gagtacaacc tgtggagtcc taaatgtctg tgaggcagtt |
| 5641 | gctagactgc accctcattt ttcccccagg aaagggcagc tgggatgaga gccagaagga |
| 5701 | gagagagctg ccccaacctt tgagaagcca gagtctggag tccaatttcc caaagaagca |
| 5761 | gagttttttg tgtgaggcag cacaaacccc acactgaata ccagcaaagt tcatttatga |
| 5821 | agtgaagttg ggactcagct ggctttagtg ggccaaaggg aagcaacccc attctcttca |
| 5881 | ccatacaccc ttttcctgca tttattcatt caccaaacct tttgatccac aaataaacta |
| 5941 | caagttctga gaggttcaaa gatgagtcag atttggtccg ttgagctcca gggggagaaa |
| 6001 | tgcagtgagg gaaaagattt gtaaaacgac gtacaatatg aagtgataag tgctaataac |
| 6061 | agaagtacaa agagaggggc caatgcaaag gaacaatgct agtagcttcc tggagcagca |
| 6121 | gccactgcgg gagctgaaac attcctaatc ttcccaagga agggcaccac ccaaaacaaa |
| 6181 | tttcctggcc aggaccagcc tatggtaaac gagtatgctt tgataccctg aagcccttga |
| 6241 | gatcaagacc ttataatctg gaggctcaac ataaggaatg ctttctacat atgtgccagt |
| 6301 | aatctctagc tctatgatgc aaataaatct aaggaagcaa gagactttca ggggatgaac |
| 6361 | cccttaaagg atggaagtag tcgtgcatcc tatccttccg tcagaaccca gcagatcatt |
| 6421 | tccctagtta tagaaacatt tgagtcttta ccccttgcca tattgacaaa gctcttaatt |
| 6481 | ggcttgacct atcacattgc tagatataaa ggctacaatc cctagactaa gaagtaggtc |
| 6541 | tccagttgaa gtagggagtc tcagtcaatg taggcagagt acaagaccct acagcctgct |
| 6601 | ctctcacctg ccatcgtaca gaccagcttt taggggagcc aagttgggat actcaatccc |
| 6661 | aacttttttc cttctcttcc atctcacata caggaaacct tacgagagag gattaggggc |
| 6721 | ctgaaaaagc tgacaagacg gcaaatatgg gaagtggagc cagtgctgag gacaaagaac |
| 6781 | tggccaagag gtccaaggag ctagaaaaga agctgcagga ggatgctgat aaggaagcca |
| 6841 | agactgtcaa gctgctactg ctgggtgagt gagatgggaa gatgagccag agaaggcagg |
| 6901 | ggtccttcct actttcctga agggttggtg ggttctacct caccccatgg gaaggaaggg |
| 6961 | tggcaggtca tttttcctct cctctacaca gtctggctag gggatcagga gatctagagc |
| 7021 | tgagttaata tggggcctaa acagccaccc caaggggatc cagaatgcca aggctattcc |
| 7081 | agagtttttc tactcttgag cgaggaatag tgcaagggcc accatgaacc cacccatttc |
| 7141 | caaatctaac caaacctaac acatcctttt ggcttcaagg accctggact tgcagactgt |
| 7201 | acccaatctg cagagaactc taagccaaga aatcagaaga gaacaggacc ttccctcacc |
| 7261 | aacaggctca caagtcccac catacagtca gtgccaacag taccagagat agtcccactg |
| 7321 | gtttttgcca agtatagtgg ttccttcttg ctttcagtaa aaactttgga agtagggggc |
| 7381 | tctgaggaag gaggaatggt gtctttatgt acagcagtcc cttcctggct ctctattcaa |
| 7441 | tagctgcctg caaagctcct gccagatgga aggttcatca acttgatgag ctcctaagca |
| 7501 | gatcactggt ctgtgctgag aaaataaaag cacctcaatt tgtcagggaa attgatcaca |
| 7561 | gctgtaaata aaaccaagac aagaacattt gagacacgtg gcttaggaaa acaaaccact |
| 7621 | ggtaccacag aagtagggta gctggagcag gtagggtcta cgtagcagaa gattagatgc |
| 7681 | ctgagctggg tttccaagcc cccataaggg atctgggagc tgacgcacta ggctaaggca |
| 7741 | ccttcttttc ccccagctga tctgtggcac agtcgtaagg acacactaaa ggagcatatc |
| 7801 | tttgtaagct ggaccagact ctaaggagcc caggaggtta cgcaggggga aacagagatg |
| 7861 | gtggggccac tgagagatct tttaagccta agcagatttc ttctacattc aggataagct |
| 7921 | gcttagaggg aacaagcaca agcgaaatag gaggagttcg aggcattagg gtagtataaa |
| 7981 | ctcagtacta gaaggtatga gtttttgatg gagagagcag agtgtgaatg aggacattag |
| 8041 | gacacattag tcaataaagg gaacccactt agccccatcc aagaccaggt tgagcaacat |
| 8101 | ggtgaaaacc tgcctctacg gaggggcggg gggaaatggc cggatgcagt ggcgcacgcc |
| 8161 | tgtaatccca gcgctttggg aggccaagat gggtggatct acctgaggtc aggagtttga |
| 8221 | gaccagcctg gccaacatag tgaaacccca tttctactaa aaatacaaaa attagtcagg |
| 8281 | catggtggca tgcgcctgta gagtcccagc tactcaggag gctgaggcag aagaattgct |
| 8341 | taaaacccag gaggcagagg ttgcagtgag ccaagatagc accactgcac tccagcctgg |
| 8401 | gagacagagc aagactgtct caaaaaaaaa aaaaaaaatg tagtcgggca tgatgtcgca |
| 8461 | cacctggaat cccaactact caggagactg aggtgggagg atcacttgag cctgggaagt |
| 8521 | caaggctgca gtaagccgag attgtgccat tacactccag cctgggctac aaacctgaga |
| 8581 | ccctgtctca aaaaaaaaaa agggaaccag caaggtgatg aaaatatagt tattttggtg |
| 8641 | aaatgatcca gctctctcca atcctacccc aagatcactc tcctgagtca aaaataggca |
| 8701 | gagaggagaa atgttaaagg actccccctg aatctgttag tggttttcaa caggaagatt |
| 8761 | ttgactgcca gtggtcattt ggaaatgtct ggagacactt ttggttgtca taactaggga |
| 8821 | agtgggatgc tattggcctc tagtgggtag aggccaggaa tgctgctgaa catcctacca |
| 8881 | tgcatgagac agcccaaact aaatagtact gaagttaaga aactgctcta aatccaggtt |
| 8941 | gaatggcctg agctcaagcc tgccagaaat tgagggcagc agtcatccct atgtattctc |
| 9001 | ccctaacaag acccccaagc aagcagtggc tctgacttct cccaggccat ctcctggaag |
| 9061 | gctgaggaga actggtggaa atcgaaagca taagcatttt tccttccagg tgctggggag |
| 9121 | tcaggaaaga gcaccatcgt caaacagatg aagtgagtag aaacaaagcc ccaaaagaca |
| 9181 | agatagggtg aagaagtcag tacagccagt ggaggtattc aaagtgaaag gctctttagc |
| 9241 | ctcaaggagc ccaggtataa aggatctgat tccaatgccc ctcatctgta ccccttgtcc |
| 9301 | ctcaccctcc actttgagaa agcagtagca acagagagat aggattctta tgatccttta |
| 9361 | aataccccca aattcctaat cccttaggtc tggttactca ggtccagcta aagacagagt |
| 9421 | gtctgcccct tgcaggatca ttcaccagga tggctattca ccagaagaat gcctggagtt |
| 9481 | caaggctatc atctatggaa atgtgctgca gtccatcctg gctatcatcc gggccatgac |
| 9541 | cacactgggc atcgattatg ctgaaccaag ctgtgcggta tgtgattact attatgtggt |
| 9601 | taagggtgga agcagaaagg ctagcaagaa gaaacatacc agaggccaac aaactatatg |
| 9661 | gaaagatggg taagaaaaat agtaactaaa atcccacctg ctgggtggga tctcactgct |
| 9721 | aggtgtactg tgtataccat ctcaaggccc tttacccatc tacgcagtaa gaccttaagg |
| 9781 | tagacggtac ctttctaatt agcctcattt tacacatgga aaaactgaag ttcagagaag |
| 9841 | ttaattaact gcccaaggtc atgtagataa atagcagaga ctgcatttgc attgggctgc |
| 9901 | ttgactacaa agctgaatat ttttcataat acaccacaat tatgcagtcc tggagaagta |
| 9961 | agcaacatcc acccttattt ttactgggac agggtcttcc tctcttaccc aggctggaat |
| 10021 | gcagtggtgt gatcatggct cactgcaatc tcaccctccc aggcttaagt gattctccca |
| 10081 | cctcagcccc agtaggtggg accacaggtg tgcaccacca cacccagcta ttttttaaaa |
| 10141 | tttttttgta gagaaggggt cttgccatct tgtctaggct ggtgtcaaac tcctggcctt |
| 10201 | aagcaatcct cctgcctcag actccgaaag tgttgagatt acaggtgtga gccaccgtgc |
| 10261 | ctgtcctcaa gccctacttt ataacacata tttacaaaat aaagctgctc acagcttcct |
| 10321 | ctgctttttt actttaccat ttaaattgct acccttgggt tcctggccat ggaacttttg |
| 10381 | taagtgaaat ccctacttct agtactggac tttttctagc tttcaatcgc tgaagaacaa |
| 10441 | aggtgatggg tccctgtctt ctatcttgtc tatttcatgg tcaatctggc ctttacattt |
| 10501 | tcagtctctc tgcagcagag gacccacgtt ttgtggcggt aaactttcag actaaggatg |
| 10561 | gaagccagaa actccagaga ggaaatacct gggacccctg ccccgcttta gacacacccc |
| 10621 | agggctgggt atagtgccac cctctgcatc tggggagcat actcaaaaat tcaacagtat |
| 10681 | gttttcttac atagaatctt cactggatac tgcttccatc ttaggtcttc gtagtaatgt |
| 10741 | aaaatgattt ccattcagca gtattcccag aagcctcctg gaaagctatt actcctgtga |
| 10801 | agttcttaac caggtttctg cattacagga tgacgggcga cagctcaaca acctggctga |
| 10861 | ctccattgag gagggaacca tgcctcctga gctcgtggag gtcattagga ggttgtggaa |
| 10921 | ggatggtggg gtgcaagcct gcttcgagag agctgcagaa taccagctta atgactccgc |
| 10981 | atcttagtaa gactgactgg tgagagggtg ggttgatgct taagcaatct tctagccagt |
| 11041 | cttctctctg gttgggagaa acctcaccca acccaaaatt tcaggcattg aaagctggag |
| 11101 | acccagactg aattcagcct gtggatctgt tttgttaggc ttcagcaatg ttttgaattt |
| 11161 | aatgctatgg ggagatctgc cacagttgtc atgactttct attgctttac actggctcac |
| 11221 | tacagcctac aaggctgaag atgcaaaatt caaaagacgt gttctgccta gtagccctca |
| 11281 | gccctgttct cttaattgag gtgtacacta agcctctcct tccagaaccc taagatagcc |
| 11341 | ctgcagttct gtcatacact ctcgcaggag taatgtgatt catgatggct ctttcagggc |
| 11401 | agaattttct atctggtaat ctaggatcta cttaggccac taagcagaaa tgagtctcct |
| 11461 | acacctagag catcttatgt cacgatgcca aggcagggga cacaaactac aagggcaaac |
| 11521 | caagggaagg tggtcaggaa ttagtaccat cattgcaaag ctgcttccaa aaagctaagg |
| 11581 | gacataattt atcaatccta cctcagacag tatgggatca tagacaagta tgggcttcaa |
| 11641 | agtcaaacct gagtttattt aacctaacca tctgtctcct aatcttgtaa taatgctatc |
| 11701 | acttactgtt cccggagcac tcaaagccca gtcactgcta ggtgtactgt gtataccatc |
| 11761 | tcaacgccct ttacccatct acgcagtaag accttaaggt agatggtacc tttctaatta |
| 11821 | gcctcatttt acacatggaa aaactgaagt tcagagaagt taattaactg cccaaggtca |
| 11881 | tgtagataaa tagcagagac tgcatttgca ttgggctgct tgactacaaa gctgaatatt |
| 11941 | tttcataata caccacaatt tctcttgacc ctagccaagt atttaactct tctgaatttc |
| 12001 | tccatctgta aaatggggat atgaaacttg atggcttagg agtttaaacg agaatctgaa |
| 12061 | cattaaaatg cctggcatac aataggcact tgtgttactt gtacttccct cctcctctca |
| 12121 | gtcatctcag ttcacatctg ccttcctctt gccttttttt tttttggaga cggagtctcg |
| 12181 | ctttgtcacc caggctggag tgcagtggcg caatctcggc tcactgccag ctttgcctcc |
| 12241 | cgggttcacg ccattctcct gcctcagcct cccaagtagc tggggatact ggcgcccgcc |
| 12301 | accatgccca gctaattttt tgtattttta gtagagatgg ggtttcaccg tgttagccag |
| 12361 | gatggtttcg atctcctgac cttgtgatcc gcccgcctcg gcctcccaaa gtactgggat |
| 12421 | tataggcgcg agccaccgtg cctggccacc tttttttttt ttaaaaaaaa taactttttc |
| 12481 | ttttttaagg taactttcaa acatacaaaa gtagaaataa cataatgaat taccttcaac |
| 12541 | agttatcaac tcatggccaa tcttatttct ttttacctcc attccctact ggattatttt |
| 12601 | gaagcaaatt ccaggtatca tttcatccta aaaatgtcat tatggatctc taaaatattg |
| 12661 | acccttttta taaaaagaat ataatcacac ccaaatagtt aacaattcct taatagcatc |
| 12721 | aaatatcacc aagtcggtgt tcaaatttcc ccagttatct caaatgtttc ctccccagtt |
| 12781 | tgattcagga tataaacaaa gtccatgtgt tgcattggtt catgtcttta aagtctcttt |
| 12841 | caatttactg cctcttgatg gaacttttag cacccactaa atctcattca aaaatctaca |
| 12901 | aaagcttgca gtcagggcaa acccaaggaa aacttatcta acttgattta aatgcgctgg |
| 12961 | aactccagcc tcaaatgggc ttgtccctca gtccatcccc catttcctct agttaatgac |
| 13021 | atatgaaggt ttgaaggaaa gaatcctaaa tttaacaata tggcttctag tcctgatttc |
| 13081 | tatcaccaaa ttagctgtgt gactttgggt aagatacctt ttacgtctct tagcctcgtc |
| 13141 | tgtggaaaat ttgtcattga tgctaatcac ttttctctag ctacctgaac caattagaac |
| 13201 | gaattacaga ccctgagtac ctccctagtg agcaagatgt gctccgatcc agagtcaaaa |
| 13261 | ccacgggcat cattgaaacc aagttttccg tcaaagactt gaatttcagg taagtgcatg |
| 13321 | gttccctagg gcatctcgga tacacgtgtg gaatcctgaa aaggggcaca ggcaggcttc |
| 13381 | ttggcctttg gtgaagccta atctaaatta ctgttctgct tctccccctt ttcctctccc |
| 13441 | acttcacggg gtgaccatca aaggcatata ggtcgtatgt tgggcatacc tatgaaaata |
| 13501 | gctgcttctt ccctcaggat gtttgatgtg ggagggcaga gatccgagag aaagaagtgg |
| 13561 | atccactgct tcgagggagt cacctgcatc attttctgtg cagccctcag tgcctatgat |
| 13621 | atggtgctgg tggaagatga cgaagtggtg agtggccttt gcatcaagca gctttggtag |
| 13681 | aacaagttct ccccatgacc ctttctctaa gccttgtgtc actctactgc cccaacttag |
| 13741 | gtaatttcag tctagcagcc ctccagcaga ccaatcaatg tctcatgcaa ataattctaa |
| 13801 | aaaacaactt cttctgcagg ttctagatta gcattttaga gctccaaatt tactgacagt |
| 13861 | gagcttggtc tcaaattaga catctaagta tcacttggac atcacaaagc tcataagagg |
| 13921 | aattgagtgc aaagagataa gggaccatca actaggcaaa gcaaaggagt tacacttagt |
| 13981 | actctcccaa attgcctaag gaaggagatg aaaatgacag aacagagaaa ataacatatg |
| 14041 | atatgaatct tcattgcaac ataatagaag ggttgagcta gtaaccccac ttaggaggct |
| 14101 | aaaaatgtac tgtccgtagg agtttaagga gagactggca gaccagcttt ctctcatgcc |
| 14161 | aattaaattg gcagctggaa gactacccaa gagtggttct ctttagcctg tagaattctg |
| 14221 | taggacagga gttctatagg acaagtgtta gagcccagcc agtttctgaa tttgggaaag |
| 14281 | gttagaggtg agaaaaacgt taatttcacc caagcatctg ctttctgaat ttgggaaaag |
| 14341 | ttagaggcgt gaaaaacgtt aatttcaccc aagcatctgc tcacaaatgg aggtccacac |
| 14401 | cttgctgatg acctctgaat ttgggaaagg ttagaggcgt gaaaaatgtt aatttcaccc |
| 14461 | aagcatctgc ttacaaatgg aggtccacag cttgctgatg aaagggatac tcctatccct |
| 14521 | tgccacagct tgttctcttc ccttcccttt ggtagtttta acttcacatt agagcactct |
| 14581 | gaatatcgtc taatcaaaat gtcttacaga gctattcact tcccatcttt aagcctaaag |
| 14641 | attacagtct atgagacttt ccatctttaa ccctaaagtc tatgagtcta tgaggtttat |
| 14701 | taaagtctat gagacattaa taaaacaagt ctatgagacc ttaaagggtt gtacaggagt |
| 14761 | attatgggga aaaagccaca aatgggattg ttcttgcttt attagataag tagactgaac |
| 14821 | gcaagtcagc tgatagtata cttcaaaacc ctaaagacct gctccctaaa agcaagctgg |
| 14881 | gctggggcaa tgggcagcct ctgcagatat gcagcccgac ttctcgctaa gtagcaatca |
| 14941 | gagaaggaaa tgagagagca gaaatgcttg tggtatggca ctgggaaatt ctctaactct |
| 15001 | caccatgtgg cagcaggacc aaagtagccc aaactgagat ctgggacccc atgaaagaag |
| 15061 | cctatcaaaa tcatcctgga gatgcatatg ggcacatgct aacttgggcc tgtttcaacc |
| 15121 | cattatcagc actacttata aaatgtcaag ttctcagttg catcctggct gctaaagatc |
| 15181 | tgcataacac attatagacc tatatgccag ccactatcat ggacaatata catacacaat |
| 15241 | ctcatttagc tttcattgta accctataag ataggaaaac agactcagaa aaagctcaat |
| 15301 | aattttccac aagtcacaca gctattagaa agatagggaa ctagaatgat cccacatctc |
| 15361 | tctggcttta ctctggtaca ttgagataag ctgttctctg tcttgctttt tacatttgga |
| 15421 | gcactggtct ctccaagggg aacaagagca ggaagtaggt agatattcta taagccaaat |
| 15481 | ctgatatttc caatggtgtt tcctcttact agaatcgtat gcatgagtct ttgcatctgt |
| 15541 | tcaacagcat atgtaaccac aaattctttg cggctacttc cattgtcctc tttctcaaca |
| 15601 | agaaggacct ctttgaggaa aaaatcaaga aagtccatct cagcatttgt tttccagagt |
| 15661 | atgatggtaa gtgtcagggg ctggaaataa taataatgcc ttttagtaga gactggcaat |
| 15721 | tgtctcattt tttaggccaa gatgacacaa aggaacttaa gggagaacct tgggcacagt |
| 15781 | tacagggttt aaattcagat actctggaat acagcaggca ttagatgcag gagagccact |
| 15841 | gacttcatat gatacctact gaaaaccaaa ggtggaaaga cacctctcct caatttcttt |
| 15901 | tcaactaaag tgagaaacac tggagtgcaa tagagaatct tccctccaaa aataggcccc |
| 15961 | caactgctgt tgtctaataa catttcaagg atcaagtcaa tcacctaaag tgagtcagca |
| 16021 | actaacaagg gttcatttat tctacttttt cactattttt ctggaaaacc aggtaacaac |
| 16081 | tcctatgatg atgcggggaa ttacataaag agccagttcc ttgacctcaa tatgcgaaaa |
| 16141 | gatgtcaaag aaatctacag tcacatgacc tgtgctacag atacacagaa tgtcaaattt |
| 16201 | gtgtttgatg cagttacaga tattatcatc aaagaaaacc tcaaggactg cggcctcttc |
| 16261 | taatcctcac cattcctcag gtataagttc tataaacagg cttggaatct gggtaattaa |
| 16321 | aaacagaaaa ttatagtcaa tataccatga catgaagaat gaatccattc tttggagatg |
| 16381 | gagtatacat gactgcaact gtatttcata cgttcttttc aaagtgggat agctattgca |
| 16441 | gcttaaagag cacaggccag tagttagaag accccccagg ttccagtact ggttttccaa |
| 16501 | cttaatacaa aactgtgaat acttta |

It will be appreciated that various embodiments of the present disclosure have been described herein for purposes of illustration, and that various modifications may be made without departing from the scope of the present disclosure. Accordingly, the various embodiments disclosed herein are not intended to be limiting.

### Retinal Organoids

In one embodiment, retinal organoids are cell clusters that mimic the cellular ultrastructure and function of retinal tissue. The cell clusters form isolated, three-dimensional (3D) structures.

Methods for visualizing these organoids include, but are not limited to, optical microscopy, electron microscopy, live microscopy and scanning probe microscopy. In various example of optical microscopy, include bright field, confocal and fluorescence.

The retinal organoids of the present disclosure can be produced using human pluripotent stem cells (hPSCs). For example, hPSCs can be induced to develop into the different cell types present in the retinal organoid through a differential protocol discussed herein. In various embodiments, the hPSCs can be human embryonic stem cells (hESCs). In some embodiments, the hPSCs can be human induced pluripotent stem cells (hiPSCs). Non-limiting examples of hESCs that can be used include WA09, WA01, WA07, BG01, BG02, HES-3, HES-2, HSF-6, HUES9, HUES7, and 16 embryonic stem cell lines (Thomson et al., 1998, Science, 282, no. 5391:1145-1147). A registry of contemplated human embryonic stem cell lines can be found at NIH Human Embryonic Stem Cell Registry.

The retinal organoids can be produced, in vitro, to be wild-type retinal organoids or can include naturally occurring genetic mutations or engineered genetic mutations.

### Disease Models

Disorders of the retina included, for example, retinitis pigmentosa (RP). RP is a heterogeneous group of rare inherited retinal degenerative diseases primarily characterized by progressive loss of photoreceptors over years to decades. RP can be caused by mutations in >80 genes involved in the function and maintenance of photoreceptors. In one example of RP, a gene for IMPG2 is mutated and results in loss of outer segments of the PR and eventual death of rods and cones, and subsequently blindness. Compromised expression of the IMPG2 gene in this type of RP results in loss of readily visible outer segments, and can be easily diagnosed based on a lack of visible hair-like outer segments on the IPM surface. Another disorder includes retinoblastoma.

In various embodiments of the disclosure, retinal organoid can include one or more genetic mutations; genetic mutations in one or both of the alleles of a gene; and/or genetic mutations in one or more of genes. There are many genetic diseases caused by well characterized and reproducible genetic mutations.

### Therapeutic Treatments

The retinal organoids of the present disclosure are designed to produce a response to a restoration of function of the one or more gene mutations.

In one example, gene function is restorable by administration of a therapeutic treatment to the retinal organoid model system. In various embodiments of this example, the therapeutic treatment can include, but is not limited to, treatment with a protein, a virus, an RNA molecule, a DNA molecule, a small molecule, a gene editor, a base editor, an RNA editor, a small molecule targeting DNA/RNA, or a cell therapy, and any combination thereof. In several non-limiting examples, the candidate therapeutic treatments include gene augmentation, genome editing, base editing, RNA trans-splicing molecules, antisense oligonucleotides, nonsense read-through drugs, and others.

In various non-limiting examples, a gene, base, or RNA editor can include CRISPR, cytosine base editors (CBEs), adenine base editors (ABEs), TALEN base editors, zinc finger nucleases, antisense oligonucleotides, RNA trans-splicing molecules, and others.

In various non-limiting examples, contemplated cell therapies include stem cell transplantation.

The therapeutic treatments can be administered to the retinal organoid model system using any suitable method. This includes, but is not limited to, nanoparticle drug delivery, membrane fusion, lipofection, ribonucleoprotein delivery, electroporation, local injection of the therapeutic treatment into the organoid, or addition of the therapeutic treatment to the media surrounding the organoid.

### Method of Screening

In addition to retinal cell lines and retinal organoids and methods of making the same, the present invention provides a method of screening a candidate agent to determine suitability for treating a retinal tissue defect of interest, comprising administering/contacting the candidate agent to the organoid of the present invention (i.e., organoids with 3-dimensional retinal cells) obtained using the methods according to the present invention, and determining the effect on the organoid. Whilst it is envisaged that candidate agents would be administered to the organoid it is also conceivable that the agents could be incorporated during the production of the organoid to understand the effects on the development of said organoid. According to this aspect, a candidate agent, e.g., a candidate therapeutic drug, can be screened for having an effect on organoids which have a known mutation, which can be introduced, in particular, the present invention provides investigations in mutations in causing retinoblastoma and allows the screening of pharmaceutical agents, which can affect the mutations, e.g., compensate for the insufficiency or overexpression in the mutated gene. A positive candidate drug could be a compound, which restores normal cellular development.

Of course, it is also possible to screen candidate agents, e.g., candidate therapeutic drugs, to have any effect on normal tissue as well, without a mutation, which leads to an aberrant development. Thus, in yet another aspect, the invention relates to a method of testing a candidate drug for physiological effects, comprising administering a candidate drug to an artificial culture/cell line culture and determining an activity of interest of the cells of said culture and comparing said activity to an activity of cells to the culture without administering said candidate drug, wherein a differential activity indicates an effect.

The present invention also envisages that the organoids, or a cell derived from said organoids, can be used in a drug discovery screen; toxicity assay; research of tissue embryology, cell lineages, and differentiation pathways; gene expression studies including recombinant gene expression or gene expression, such as using an inducible Cre-based expression of cDNAs or CRISPR components 3′ to a Lox-Stop-Stop-Lox element and 5′ to a 2A peptide linked fluorescent protein marker; research of mechanisms involved in tissue injury and repair; research of inflammatory and infectious diseases; studies of pathogenetic mechanisms; or studies of mechanisms of cell transformation and aetiology of retinal disease.

The organoid of the invention, or a cell derived from said organoid, is also envisaged for use in medicine. For example, said organoid, or a cell derived from said organoid, could be used for use in treating a retinal disorder, condition or disease such as diabetic retinopathy, retinopathy of prematurity, macular degeneration, Usher syndrome, Stargardt disease, Retinitis Pigmentosa, age-related macular degeneration (AMD) and inherited retinal dystrophies (HRDs). One option is that said organoid, or a cell derived from said organoid could be used in regenerative medicine, for example, wherein the use involves transplantation of the organoid or cell into a patient.

### EXAMPLES

The Examples that follow are illustrative of specific embodiments of the disclosure, and various uses thereof. They are set forth for explanatory purposes only and should not be construed as limiting the scope of the disclosure in any way.

### Example I

### Introduction

Cone photoreceptors are needed for color vision and are impaired in retinal diseases such as cone-rod dystrophy, retinitis pigmentosa, Leber congenital amaurosis, and retinoblastoma (Mustafi et al., 2009; Xu et al., 2014). Modeling of human cone development and disease in animals is challenged by human-specific cone development features (Singh et al., 2018). Human pluripotent stem cell (hPSC) derived retinal organoids (ROs) closely recapitulate human retinal development with similar developmental timeline, cellular composition and laminated structures (Aparicio et al., 2017; Bell et al., 2020). Combined with CRISPR gene editing, ROs provide opportunities to build cone disease models for mechanistic studies and therapeutic screening and are a source for retinal cell or tissue transplantation (Gasparini et al., 2019).

hPSC-derived ROs with cell type-specific fluorescent reporters may be used to monitor a cell's normal and disease-related behaviors. Photoreceptor reporter lines have been generated by introducing a CRX-GFP cassette (Kaewkhaw et al., 2015) or a mouse Crx-mCherry cassette (Gagliardi et al., 2018) into the AAVS1 locus. A rod reporter line was made by replacing the NRL coding sequence with EGFP (Phillips et al., 2018); cone reporter lines were produced by inserting a mouse cone-arrestin (mCar)-GFP cassette (Gasparini et al., 2022) or inserting T2A-mCherry at the C terminus of GNGT2 (Nazlamova et al., 2022); and a retinal ganglion cell (RGC) reporter line was produced by inserting a P2A-tdTomato-P2A-Thyl.2 cassette at the C terminus of BRN3B (Sluch et al., 2017). Additional lines reporting multiple cell types include a SIX6-GFP/POU4F2-tdTomato double reporter line that separately labels all retinal cells and RGCs and a VSX2-Cerulean/BRN3b-EGFP/RCVRN-mCherry triple reporter line that differentially labels retinal progenitor cells, RGCs, and photoreceptors (Lam et al., 2020; Wahlin et al., 2021). These lines have been used to investigate retinal morphogenesis, improve organoid differentiation, and purify specific retinal cell types for transcriptome profiling and cell transplantation.

To build a platform with which to study cone development and diseases, provided herein is a human cone reporter iPSC line in which EGFP is specifically expressed in both immature and mature cones with minimal disruption of normal cone development. GNAT2, which encodes the cone-specific α-subunit of transducin, a G-protein that couples visual pigment opsin to the cone phototransduction cascade (Morris et al., 1997; Morris & Fong, 1993) was selected. Prior studies demonstrated that Gnat2−/− mice exhibit complete loss of cone phototransduction without changes in rod phototransduction or in cone or rod morphology (Ronning et al., 2018). In human retina, GNAT2 expression is limited to cones, controlled by a cone-specific promoter, and initially induced following the early cone lineage determinants RXRG and THRB but prior to mature cone markers ARR3, OPNISW or OPNILW (Hoshino et al., 2017; Morris et al., 1997; Welby et al., 2017) (data from (Hoshino et al., 2017)). However, unlike RXRG or THRB, GNAT2 does not downregulate in adult cones (Hoshino et al., 2017; Welby et al., 2017). A similar onset order was observed in CRX-GFP+ photoreceptors in human ROs, with GNAT2 first detected at day 37 (Kaewkhaw et al., 2015). These features suggested that the endogenous GNAT2 promoter linked to a fluorescent protein may serve as an ideal cone-specific reporter.

In principle, cell type-specific fluorescent reporter lines enable continuous or episodic live imaging to characterize developmental and disease processes. Episodic long-term imaging of specific RO regions enables repeated monitoring of individual retinal cells, yet embedding beyond the initial six weeks was shown to impair photoreceptor development (Decembrini et al., 2020; Rashidi et al., 2022). Others have immobilized and imaged mature ROs for up to three weeks (Achberger et al., 2019), yet longer-term imaging has not been demonstrated.

Provided herein are GNAT2-EGFP cone reporter iPSC lines in which cones are robustly, specifically, and innocuously labeled with EGFP. RO hydrogel immobilization and episodic live imaging methods were established that enable long-term assessment of individual EGFP+ cone morphological changes, inner segment development, and mitochondria localization. This EGFP-GNAT2 cone reporter line, combined with the immobilization and imaging techniques, provides a useful tool to study cone development and disease.

### Results

GNAT2-EGFP iPSCs

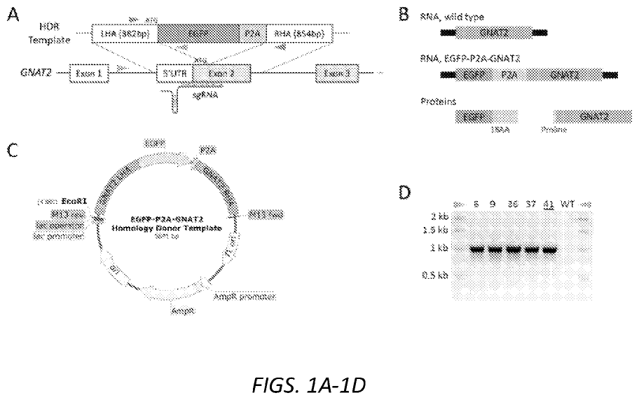

To assess the suitability of a GNAT2 cone reporter, GNAT2 expression was compared to that of other potential cone markers in human fetal, adult, and retinal organoid scRNA-seq datasets. In a combined human fetal retina, adult retina, and early-stage RO dataset produced via 3′ end-counting, GNAT2 was mainly detected in cones from adult retina (Fig. S1B-E; data from (Lu et al., 2020)). In contrast, the deep, full-length single cell RNA-seq analysis of fetal retinal progenitor cells and photoreceptors showed robust and specific GNAT2 expression in the majority of fetal cones from post-conception week 13 to 19 (Fig. S1F, G; data from (Shayler et al., submitted)), consistent with the onset timing in bulk RNA-seq (Fig. S1A). This sensitivity and specificity compared favorably with other potential cone markers such as THRB, RXRG, GNGT2, ARR3, OPNISW, or OPNILW, which were either expressed in both cones and rods (THRB, RXRG, GNGT2) or expressed in only a subset of cones (ARR3, OPNISW, OPNILW) (Supplementary FIG. 1H-O; data from (Shayler et al., submitted)). These patterns were preserved in human ROs, where GNAT2 was detected at day 37 in CRX-GFP positive cells (Kaewkhaw et al., 2015) and specific to cones at an initial day 55 time point (Shayler, Cobrinik et al., data not shown).

To generate a GNAT2 cone reporter line, CRISPR/Cas9-mediated homologous recombination was used to insert an EGFP-P2A cassette at the N-terminus of GNAT2 in the human WTC11 iPSC derived line WTC11-mTagRFPT-LMNB1 (Allen Institute for Cell Science) (FIG. 1A, B). The N-terminal position of the EGFP-P2A cassette is predicted to enable GNAT2 translation with a single proline residue added to the N-terminus (FIG. 1B). WTC11-mTagRFPT-LMNB1 cells and derivatives express an mTagRFPT-Lamin B1 fusion protein to enable live imaging of nuclei together with other fluorescent protein markers.

Briefly, a homology donor plasmid was constructed by inserting the EGFP-P2A coding sequence between left and right homology arms (LHA and RHA) containing human GNAT2 genomic sequences 882 bp upstream and 854 bp downstream of the translation start codon (FIG. 1C). The sgRNA spanned the intended insertion site, eliminating the need to introduce a silent mutation on the homology donor plasmid (FIG. 1A). No antibiotic resistance marker was included in the donor vector to enable scarless editing. Following electroporation of the donor plasmid and a plasmid co-expressing GNAT2 sgRNA and Cas9-T2A-Puro (PX459) (Ran et al., 2013), cells were selected with puromycin, single-cell cloned, and screened by PCR using location-specific and insert-flanking primer pairs (FIG. 1A). PCR with location-specific primers flanking the LHA showed integration of the EGFP-P2A cassette with correct orientation in five of 48 clones tested (FIG. 1D), while insert-flanking primers distinguished two mono-allelic from three bi-allelic knock-in clones (data not shown). The two mono-allelic clones carried mutations on the non-knock-in alleles, whereas the three bi-allelic clones carry no unintended mutations at the knock-in junctions. Partial sequencing of EGFP-P2A-GNAT2 (hereafter referred to as GNAT2-EGFP) clone-41 (C-41) revealed no unintended mutations at any of the top five predicted sgRNA off-target sites (Fig. S2A, B; Table 1) and karyotyping revealed no chromosomal abnormalities (Fig. S2C). All subsequent experiments were carried out using this clone.

### Table 1 (SEQ ID NOs: 1-6)

Top Five Off Targets Predicted by IDT CRISPR-Cas9 gRNA Checker

| Sequence | PAM | Score | #MM | Gene | Locus |
|---|---|---|---|---|---|
| AAGACGGCAAATATGGGAAG | TGG | N/A | | GNAT2 | chr1: −109612862 |
| CAGATGGTAAATATGGGAAG | TAG | 0 | 3 | | chr7: +15609261 |
| CAGA-GGTAAATATGGGAAG | TGG | 3 | 3 | | chr10: −107997752 |
| AACATGGTAAATATAGGAAG | TAG | 8 | 4 | | chr10: +26802625 |
| ACCACGTGAAATATGGGAAG | TGG | 9 | 4 | | chr22: −26429847 |
| TAGAAGGTATATATGGGAAG | AGG | 9 | 4 | | chr4: −61246789 |

### GNAT2-EGFP ROs

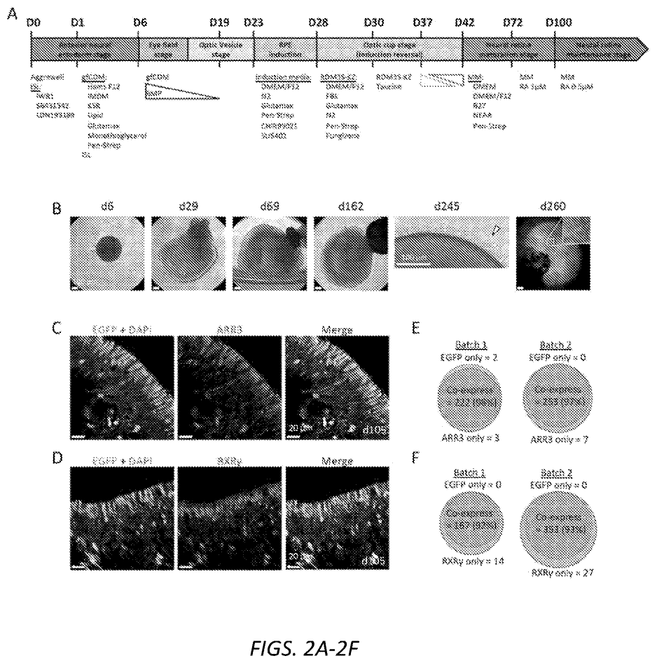

The GNAT2-EGFP iPSCs' ability to make ROs with cone-specific EGFP expression was evaluated. A modification of the Kuwahara et al. protocol (Kuwahara et al., 2015) was used improve RO consistency. Initial culture was supplemented with small molecule inhibitors of WNT signaling (IWR1) and TGF-β super family signaling (SB431542 and LDN193189) for six days, followed by addition of BMP to induce anterior neural ectoderm and eye field specification (Aparicio et al. in submission). As the parental WTC11-mTagRFPT-LMNB1 had poor early survival, the starting cell number was increased from 12,000 to 48,000, the retinal pigment epithelium induction-reversal was optimally timed from d23-d28, and long-term maintenance with retinoic acid was begun at d72 (FIG. 2A), which promotes photoreceptor maturation and long-term survival (Kelley et al., 1994; Zhong et al., 2014). During the first 30 days, ROs increased in size and adopted a laminated structure indicative of nascent neural retina. Subsequently, RO growth slowed and ROs shed cells or debris between d80 and d100. A brush border likely representing photoreceptor inner and/or outer segments was evident by ~d140 and remained visible until the latest analysis on d245 (FIG. 2B). In mature ROs, EGFP+ cells formed uneven patches occupying the outer-most layer (FIG. 2B).

To evaluate the specificity of GNAT2-EGFP expression, d105 RO sections were immune-stained with cone-specific markers ARR3 and RXRγ and assessed their co-localization with EGFP. At this age, most EGFP+ cells had elongated cell bodies occupying the outer-most layer (FIG. 2C, D). Among 227 cells from 6 sections and two ROs examined for ARR3 and EGFP expression, 222 co-expressed both, three expressed ARR3 only, and two expressed EGFP only (FIG. 2C, E). Among 181 cells from five sections and two ROs examined for RXRγ and EGFP expression, 167 co-expressed both, 14 expressed RXRγ only, and none were EGFP+ and RXRγ negative (FIG. 2D, F). These results are consistent with the sequential onset of RXRγ, GNAT2-EGFP, and ARR3 expression during cone maturation (Welby et al., 2017) and confirm the specific and robust labeling of cones in GNAT2-EGFP ROs.

### High-Resolution Live Confocal Imaging of Cone Maturation

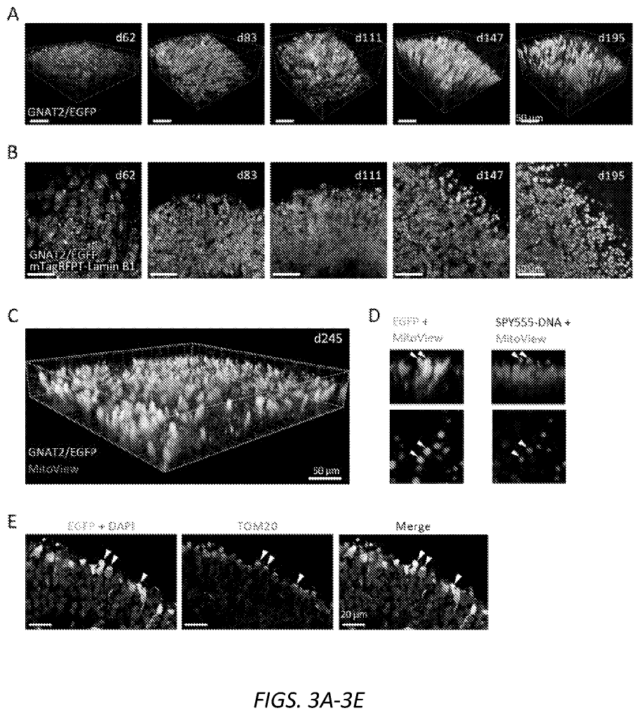

The feasibility of high-resolution live imaging of GNAT2-EGFP ROs to monitor development of cone cells over time was assessed. Episodic live confocal imaging was performed on GNAT2-EGFP ROs and z-stack images were captured at different maturation stages, with cones represented by cytoplasmic+nuclear EGFP and nuclear membranes represented by mTagRFPT-Lamin B1 (FIG. 3A, B). From d62 to d111, EGFP+ cell bodies elongated and gradually populated the outer-most organoid layer. By d147 they developed more mature cone morphology, with the appearance of inner segments and pedicles similar to those in older ROs. By d195, cones retained similar morphology and cell bodies were often displaced away from the outermost layer as previously described (Gasparini et al., 2022). Unexpectedly, the mTagRFPT-Lamin B1 nuclear envelope signal was weaker for cones than other retinal cell types, possibly related to the slow turnover of Lamin B1 protein in photoreceptors (Razafsky et al., 2016).

Live confocal imaging also enabled assessment of organelle development. At d245, MitoView staining showed that mitochondria coalesced at the mature cones' inner segment ellipsoid bodies (FIG. 3C, D),D), as confirmed by immunostaining with mitochondria marker TOM20 (FIG. 3E).

### Cone Maturation in Hydrogel Immobilized GNAT2-EGFP ROs

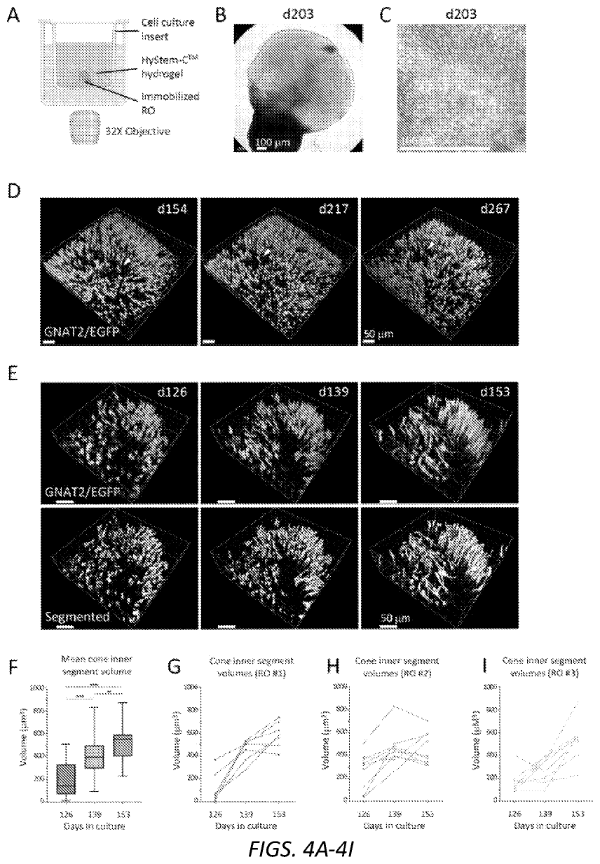

To evaluate the maturation of individual cone cells, ROs were embedded in HyStem-C™ hydrogel on Millicell™ cell culture inserts and the same organoid regions repeatedly imaged during long-term culture (FIG. 4A). HyStem-C™ is based on hyaluronic acid polymers and collagen crosslinked polymers and was chosen both because its rigidity can be tuned and because hyaluronic acid and collagen are major components of retinal extra cellular matrix and vitreous humor (Achberger et al., 2019; Hemshekhar et al., 2016) and deemed likely to be biocompatible. Experiments showed 0.25 to 1% HyStem-C™ supported long-term immobilization whereas organoids separated from 2% and 4% hydrogel. ROs embedded in 1% hydrogel at d121 or older could be cultured in this manner for at least 120 more days. The embedded ROs displayed continuous morphological changes with appearance of photoreceptor inner segment protrusions suggesting that embedding does not restrict cone growth and maturation (FIG. 4B, C). Indeed, in organoids embedded on d139, mature cones remained stable and individual cells identifiable until at least d267 (FIG. 4D).

Episodic live confocal imaging of immobilized ROs allowed one to capture developmental features of the same cells over time (FIG. 4E). As an example, GFP intensity and cell shape-based segmentation was used on the acquired 3-dimensional Z-stack images to define the volumetric change of 24 cone inner segments in three Ros immobilized ad d125 and imaged between d126 and d153 (FIG. 4E). The individual cells were within a defined region where spatial relationships were maintained. We observed the inner segments enlarged from a mean 193 μm³ to 523 μm³ (p<0.0001) (FIG. 4F), with an average increment of 12.2 μm³ per day. However, the initial inner segment size and rate of individual cone inner segment growth varied depending on the organoid and/or organoid region, ranging from a maximum rate of 27.14 μm³ per day for a cone from RO #3 to a slight decline of 0.3 μm³ per day for one cone from RO #2 (FIG. 4G-I). Taken together, episodic live imaging of hydrogel embedded GNAT2-EGFP ROs enabled long-term evaluation of cone development, such as inner segment morphologenesis and formation of mitochondria-rich ellipsoid bodies, demonstrating the versatility of this reporter system.

### Discussion

In this study, a cone-specific GNAT2-EGFP iPSC reporter line was generated and its utility for tracking individual cone development in long-term live-embedded ROs was demonstrated. By tagging GNAT2 with scarless CRISPR insertion and placing the EGFP-P2A at the N-terminus, a reporter line that faithfully recapitulates cone development with minimal effect on GNAT2 expression or protein structure was created. The GNAT2-EGFP iPSC line robustly labels GNAT2+ cones throughout RO differentiation, with EGFP detected in cone precursor cell bodies as early as d34 and subsequently in maturing cone axon terminals and inner segments. Live imaging revealed that maturing cone precursors develop inner segments and extend pedicles to outer plexiform layer between ~d120 and ~d150, coinciding with a time of rapid maturation and high glycolytic activity in the photoreceptor layer (Browne et al., 2017).

Recently, two other cone reporter human iPSC lines have been described-one generated by piggyBac mediated insertion of GFP under the control of mouse cone-arrestin (mCar) promoter (Gasparini et al., 2022) and the other generated by inserting a T2A-mCherry cassette into the GNGT2 locus (Nazlamova et al., 2022). Cone-arrestin is first expressed at a later stage of cone maturation than GNAT2, limiting its ability to label immature cones (Hoshino et al., 2017; Welby et al., 2017) (Fig. S1A). Moreover, only ~80% of cells labeled with the mCar-GFP reporter were ARR3+ whereas >95% were recoverin+ (Gasparini et al., 2022), potentially reflecting some non-cone expression. The CRISPR-based editing of GNAT2 is similar to the Nazlamova et al. editing of GNGT2, but used an antibiotic-free scarless knock-in approach so the inserted sequence contains only EGFP-P2A. Additionally, 3′ end counting and the full-length scRNA-seq analyses indicate that GNAT2 RNA is highly cone-specific, whereas GNGT2 RNA is expressed in both cones and rods and has no discernable cone-rod specific splicing or exon usage (Fig. S10) (Lu et al., 2020) (Shayler et al., in revision).

Furthermore, a protocol with which to track individual cone development was established. Live-embedding in HyStem-C™ hydrogel enabled long-term immobilized RO cultures and monitoring of cone development with episodic live confocal imaging. Immobilizing the ROs with thin layers of the hydrogel on TC inserts allows nutrient diffusion from all sides and allows RO to grow and mature with minimal restriction (FIG. 4A). Cells were tracked for at least 120 days starting at >d120. The hydrogel maintained structural integrity and transparency during the entire period, allowing repeated imaging of the same RO regions. However, live embedding might be more disruptive in younger ROs<d80 due to the ongoing rapid organoid growth and morphological changes. The biocompatibility of HyStem-C™ hydrogel was evident from the fairly consistent growth of cone inner segments and may relate to its derivation from hyaluronic acid and collagen, which are major components of vitreous humor and retinal extracellular matrix (Achberger et al., 2019; Hemshekhar et al., 2016; Tram & Swindle-Reilly, 2018). Combining this GNAT2-EGFP cone reporter with further CRISPR editing and live imaging provides a powerful tool to study cone development and diseases.

### Methods

Human iPSC Culture

WTC-mTagRFPT-LMNB1 human iPSCs (Allen Institute for Cell Science) were cultured in feeder-free conditions in mTeSR Plus media (Stem Cell Technologies, #100-0276) on Matrigel (Corning, #354277) coated 35 mm dishes. Cells were seeded at 25,000 cells per dish, fed daily, and passaged every 5 days at approximately 70% confluency. When passaged, cells were washed with DPBS (Corning, #21-031-CV) and colonies gently lifted by 2 min incubation in ReLeSR (Stem Cell Technologies, #05872) at 37° C. After neutralization with mTeSR Plus, cells were centrifuged for 3 min at 300×g and seeded at desired dilutions.

Generation of EGFP-P2A-GNAT2 Homology Donor and sgRNA Plasmids

The 882 bp left homology arm (LHA) and 854 bp right homology arm (RHA) were PCR amplified from WTC-mTagRFPT-LMNB1 iPSC genomic DNA with CloneAmp HiFi PCR Premix (Takara #639298). The LHA, EGFP-P2A, and RHA were cloned into pUC118 backbone using In-Fusion Snap Assembly (Takara #638949). The sgRNA targeting the GNAT2 start codon (AAGACGGCAAATATGGGAAG; SEQ ID NO: 7) was identified using the online crispr.mit.edu sgRNA designing tool and cloned into the PX459 sgRNA Cas9-T2A-Puro expression plasmid (Addgene #62988) (Ran et al., 2013) according to the accompanying Zhang Laboratory Target Sequence Cloning Protocol. The resulting plasmids were sequenced to confirm correct assembly. A full list of cloning primers can be found in Table 2.

### Table 2 (SEQ ID NOs: 8-32)

gRNA sequence

| | |
|---|---|
| GNAT2 gRNA | AAGACGGCAAATATGGGAAG |

Cloning primers

| | |
|---|---|
| GNAT2 sgRNA F | CACCGAAGACGGCAAATATGGGAAG |
| GNAT2 sgRNA R | AAACCTTCCCATATTTGCCGTCTTC |
| pUC118 F | GAACATTTGAGACACGTGGCTTAAGACCTGCAGGCATGCAAG |
| pUC118 R | GGTGTATGGTGAAGAGAATGGGGACTCTAGAGGATCCCCGGG |
| EGFP F | AAGACGGCAAATATGGTGAGCAAGGGCGAGGAG |
| EGFP R | ACTGGCTCCACTTCCAGGCCCGGGATTCTCCTC |
| GNAT2 LHA F | CCCATTCTCTTCACCATACAGCCT |
| GNAT2 LHA R | ATTTGCCGTCTTGTCAGCTTTTTC |
| GNAT2 RHA F | GGAAGTGGAGCCAGTGCTGA |
| GNAT2 RHA R | AGCCACGTGTCTCAAATGTTCTTG |

Primers for GNAT2-EGFP genotyping

| | |
|---|---|
| Location specific F | GCTGGCTTTAGTGGGCCAAA |
| Location specific R | CTTGTGGCCGTTTACGTCGC |
| Insert flanking F | TACAGCCTGCTCTCTCACCT |
| Insert flanking R | AGGTAGAACCCACCAACCCT |

Primers for amplifying GNAT2-EGFP off target regions

| | |
|---|---|
| Off target 1F | GCCTCTGATCTTAGTTTACTAGTCT |
| Off target 1R | ACTGACAAACAGGGAAGCCA |
| Off target 2F | TCCCAACCATAGCACTTGGG |
| Off target 2R | AGTTTGCTTTTGCCCAGCAT |
| Off target 3F | ATTAAGGCAGGCGCCAGATG |
| Off target 3R | GGAAGCAGTGAGAAAGGCCA |
| Off target 4F | GTGGGAGTTGTGGTCTTCGC |
| Off target 4R | AAGGAGGTTTATGGCGCCTC |
| Off target 5F | ATGAGCTAGATGGTGCCTGA |
| Off target 5R | AACAGAATGCAATGAAGGCTCT |

Generation of GNAT2-EGFP iPSCs

WTC-mTagRFPT-LMNB1 human iPSCs were dissociated into single cells with 5 min incubation in Accutase (Life Technologies, #A1110501) at 37° C. 200,000 cells were electroporated with 500 ng PX459 and 1000 ng of homology donor plasmid in 10 μl electroporation buffer R using the Neon Transfection System (Invitrogen) with one pulse of 1400 V and a pulse width of 20 ms. After electroporation, cells were placed in mTeSR Plus supplemented with CloneR (Stem Cell Technologies, #05888) for 36 hours and then selected in 250 ng/ml puromycin for 36 hours. After a recovery phase of 4 days, colonies were dissociated into single cells with Accutase and seeded into 96-well plates at an average density of 0.5 cells per well to ensure colonies are derived from single cells. Colonies were expanded and genotyped using two primer pairs: an LHA flanking primer pair that produces a 990 bp band from clones with correct integration of the insert, and an insert flanking primer pair that produces a single 1131 bp band from bi-allelic knock-in clones, the 1131 bp band and a 351 bp from mono-allelic knock-in clones, or a single band at 351 bp from wild type clones. The 1131 bp KI band and 351 bp WT allele band were gel purified (Qiagen, #28604) and sequenced to check for potential mutations introduced during editing. To check for potential off-target mutations, we PCR amplified ~1 kb regions spanning the top five program predicted off-target sites (IDT CRISPR-Cas9 gRNA checker) from the edited clone-41 and the unedited WTC-mTagRFPT-LMNB1 iPSCs and aligned the sequences. All PCR utilized CloneAmp HiFi PCR Premix according to the manufacturer's instructions. The predicted off target sequences and amplifying primers are provided in Table 1 and 2. The GNAT2-EGFP iPSC line was karyotyped by the Department of Pathology and Laboratory Medicine, Children's Hospital Los Angeles.

### Retinal Organoid Differentiation

Retinal organoids were generated following steps modified from a previous protocol (Kuwahara, et al., 2015; Aparicio et al., submitted). Briefly, iPSCs were dissociated on do with Accutase and resuspended in Aggrewell media (Stem Cell Technologies, #05893) supplemented with 20 μM Y-27632 (Cayman Chemical, #10005583) and “ISL” cocktail, which consists of 3 μM IWR1 (Cayman Chemical, #13659), 10 μM SB431542 (Cayman Chemical, #13031), and 0.1 μM LDN193189 (SIGMA, #SML0559). Cells were plated in round-bottom 96-well plates at 48,000 cells per well in 200 μl media to allow aggregate formation. From d1 to d5, media was changed to gfCDM (45% Hams F12 (Thermo Fisher, #11765047), 45% IMDM (Thermo Fisher, #12440046), 10% KSR (Life Technologies, #10828028), 1× Chemically Defined Lipid (Gibco, #11905-031), 0.5×Glutamax (Life Technologies, #35050061), 450 μM Monothioglycerol (Sigma, #M6145), and 1× Penicillin-Streptomycin (Corning, #30-002-CI)) supplemented with ISL. On d6, gfCDM media was supplemented with 5 nM BMP-4 (R&D Systems, #314-BP-050), followed by ½ media change on d9 and d12, ¾ media change on d15 and full media change on d19 and d21 with fresh gfCDM only without BMP. From d23 to d27, media was changed to RPE induction media that consists of DMEM/F12 (Thermo Fisher, #21331020), 1×N2 supplement (Thermo Fisher, #17502048), 1×Glutamax, 1× Penicillin-Streptomycin, 3 μM CHIR99021 (Cayman Chemical, #13122), and 5 μM SU5402 (Cayman Chemical, #13182). Starting at d28, media was changed to RDM3S-KZ, which consists of DMEM/F12, 10% Fetal Bovine Serum (Omega Scientific, #FB-01), 1× Glutamax, 1×N2 supplement, 1× Penicillin-Streptomycin, and 0.5×Fungizone (Omega Scientific, #FG-70). Taurine (Sigma, #T8691) 0.1 mM was added from d30 onward. On d30, ROs were transferred to 48-well cell culture plate pre-coated with HEMA (Sigma, #P3932-25G). From d37 to d42, media was transitioned to RO maintenance media (MM) adapted from Zhong et al., (2014): ⅔ RDM3S-KZ+⅓ MM d37, ⅓ RDM3S-KZ+⅔ MM on d40, and all MM on d42. MM consists of equal volume of DMEM (VWR, #54000-305) and DMEM/F12, supplemented with 1×B27 supplement (Thermo Fisher, #12587010), 1×NEAA, 1× Penicillin-Streptomycin, 1× Fungizone, 10% FBS, 0.1 mM Taurine, and 1×Glutamax. ROs were subsequently cultured in MM with 1 μM retinoic acid (Sigma, #R2625) from d72 to d100 and 0.5 μM retinoic acid from d100 onward. Differences from the Aparicio et al. protocol (Aparicio et al, submitted) included 1) cultures were initiated with 48,000 cells; 2) only BMP-4 was added on d6, no IWR1; 3) induction reversal was initiated on d23 for five days; and 4) RA was first added on d72.

### Immunohistochemistry and Quantification

ROs were fixed in 4% paraformaldehyde for 12 min, washed with DPBS 3 times, incubated in 30% sucrose solution overnight at 4° C., embedded in OCT compound, and cryo-sectioned into 20 mm sections. For immunostaining, slides were washed with TBS, blocked for 1 h at room temperature (2.5% horse serum, 2.5% donkey serum, 2.5% human serum, 1% BSA, 0.1% Triton X100, and 0.05% Tween 20 in 1×TBS), incubated in primary antibodies at 4° C. overnight, followed by TBS wash, secondary antibody incubation at room temperature for 1h, washing, and mounting with Mowiol with anti-fade. Samples were imaged on LSM710 confocal microscope (Zeiss) and processed using Fiji ImageJ. Primary antibodies are in Table 3.

### Table 3

| | Supplier | Catalog | |
|---|---|---|---|
| Antibody | company | number | Dilution |
| Goat anti GFP | Abcam | ab6673 | 1:400 |
| Mouse anti ARR3 | Millipore Sigma | MABN2636-25UG | 1:500 |
| Rabbit anti RXRy | Santa Cruz | sc-555 | 1:1000 |
| | Biotechnology | | |
| Rabbit anti TOM20 | Proteintech | 11802-1-AP | 1:200 |

### RO Immobilization, Live Episodic Confocal Imaging and Image Processing

ROs were subjected to live episodic imaging using a LSM780 NLO confocal microscope (Zeiss), with fitted temperature control and CO2 chamber. C-Achroplan 32x/0.85 W Korr M27 lens was used to maximize the imaging distance. Non-immobilized ROs were submerged in RO media and imaged in Lab-Tek 8-well chambered coverglass (Fisher Scientific, #12-565-338). For mitochondria live imaging, ROs were incubated with 1×SPY555 vital DNA dye (Spirochrome, #SC201) over night and Mito View 650 (Biotium, #70075) at 200 nM for 30 min prior to imaging. For RO immobilization, individual ROs were live-embedded in 100 μl 1% HyStem-C™ hydrogel (Advanced Biomatrix, #GS312) on Millicell™ 12 mm cell culture inserts with 0.4 μm hydrophilic PTFE membrane (Sigma, #PICM01250). The cell culture insert was then submerged in RO media in a 24-well plate, and media was changed following the same protocol as non-immobilized ROs. For live confocal imaging, the rim of the cell culture insert was marked at the 12 and 3 o'clock positions to indicate orientation, and inserts placed in Cellvis 24-well coverglass bottom plates. Ubiquitous autofluorescent debris on the PTFE membrane was used as points of reference for the regions of interest. Collected 3D Z-stack images were processed using Imaris (Oxford Instruments) and individual cone cells segmented using Imaris Surface function. The inner segment was manually segmented from the cell body at the thinnest connecting point and the volume recorded in Imaris. GraphPad Prism was used for statistical test with repeated measure one way ANOVA and to generate graphs.

### Example 2

### Modeling Multi-Step Retinoblastoma Genesis In Vitro With Cone Reporter Retinal Organoids

Retinoblastoma, the most prevalent childhood intraocular malignancy, originates from maturing cone photoreceptor precursors with biallelic RB1 inactivation. In explanted fetal retina, pRB-depleted post-mitotic cone precursors proliferate, followed by a 4-5-month premalignant indolent phase before re-entering cell cycle to form retinoblastoma-like masses at tissue ages similar to that of retinoblastoma in vivo, yet Rb1 mutant animal models fail to recapitulate retinoblastomagenesis with a cone cell-of-origin, likely due to human-specific cone development features. RB1^{-/-} retinal organoids (ROs) demonstrate initial cone proliferation but subsequently deteriorate. Produced and characterized herein is an RB1-null cone reporter RO model that recapitulates multi-step retinoblastomagenesis. A cone-reporter iPSC line was generated through CRISPR knock-in EGFP-P2A at the N-terminus of GNAT2 in WTC11-mTagRFPT-LMNB1. In ROs derived from these iPSCs, EGFP+ cone precursors first appear at d34 and adopt mature cone morphology at ~d120. Immunohistochemistry with cone markers ARR3 and RXRγ confirmed cone specific EGFP expression. A second round of CRISPR editing produced homozygous RB1 knockout. Chimeric organoids generated from RB1-null cone reporter iPSCs mixed with unedited parental iPSCs recapitulated pRB loss in a subset of retinal cells in an otherwise healthy retina and enabled live-imaging cell tracking in intact hydrogel embedded organoids. Bi-weekly live confocal imaging of EGFP+ RB1^{-/-} cones from d85 to d238 captured their initial proliferation followed by a pre-malignant indolence phase. The majority of the initially proliferating cones remain quiescent, with some adopting mature cone morphology. Nascent retinoblastoma-like foci were detected on several chimeric organoids after d281, a tissue age that equates to the first post-natal month when early retinoblastomas typically emerge. The EGFP+ retinoblastoma-like cells expressed cone markers and proliferation marker Ki67. In summary, a human retinoblastoma organoid model was regenerated that recapitulates the cell-of-origin and timing of multi-step retinoblastomagenesis, paving the way for mechanistic studies and therapeutic screening.

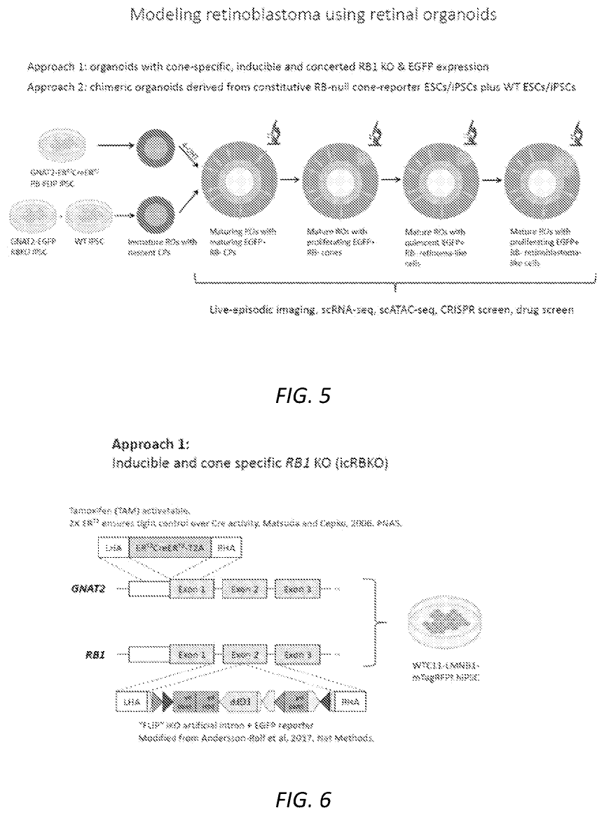

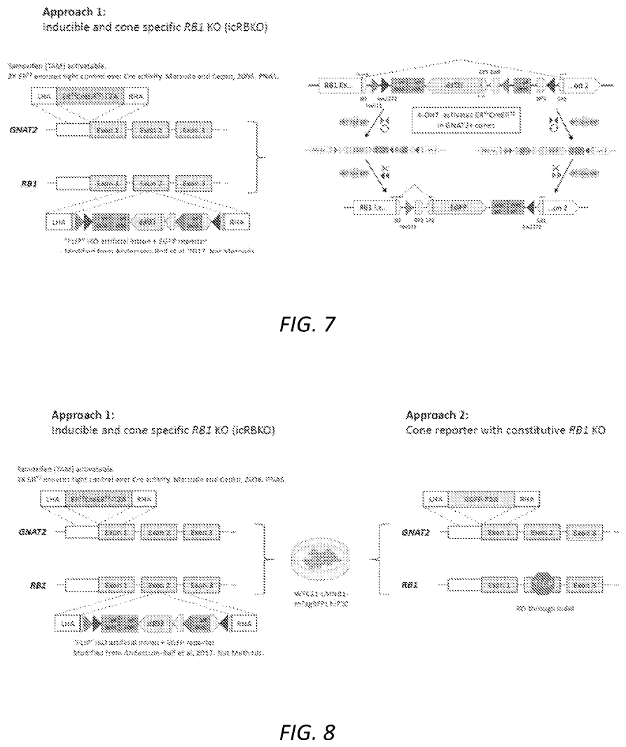

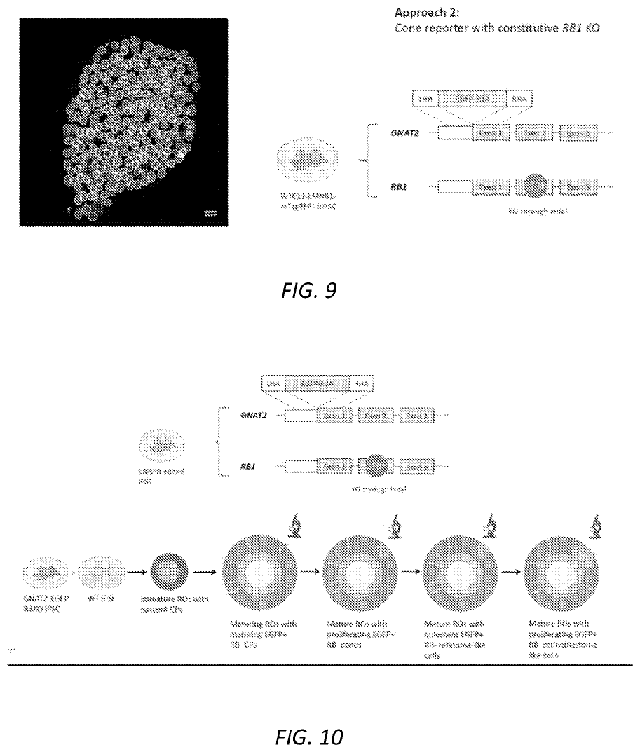

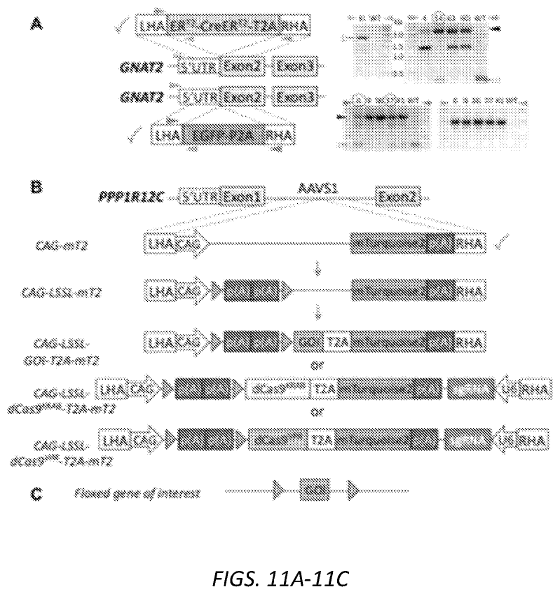

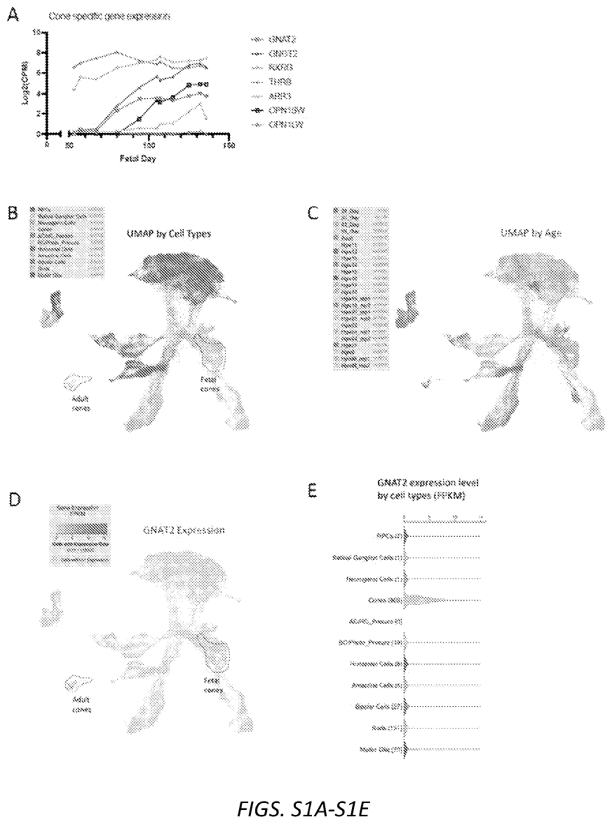

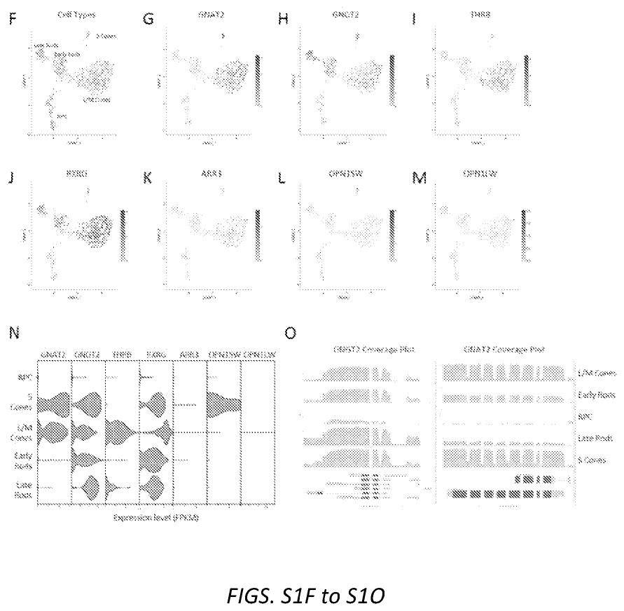

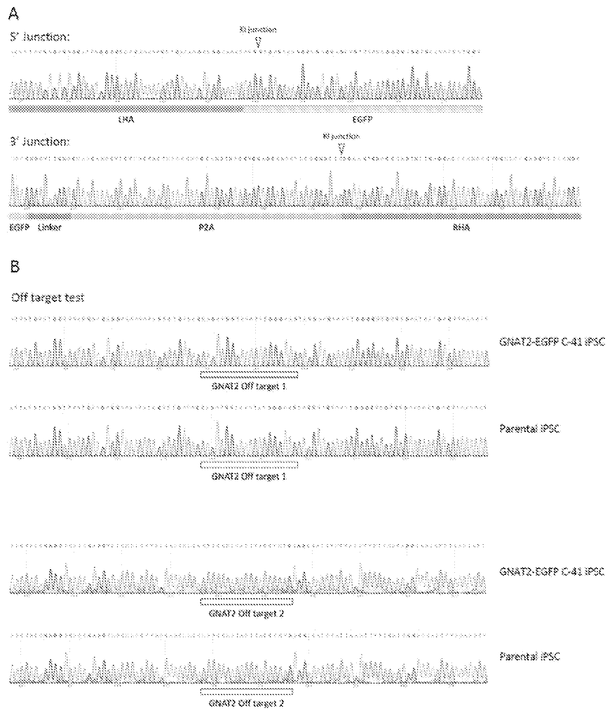

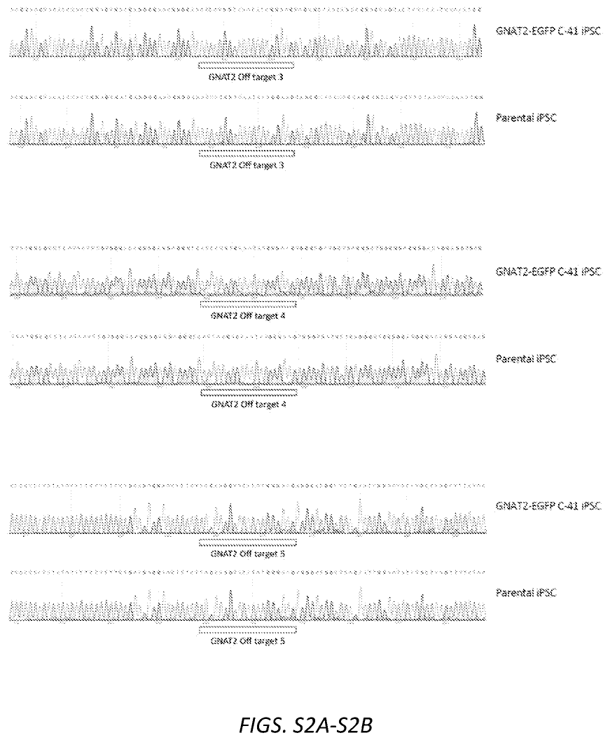

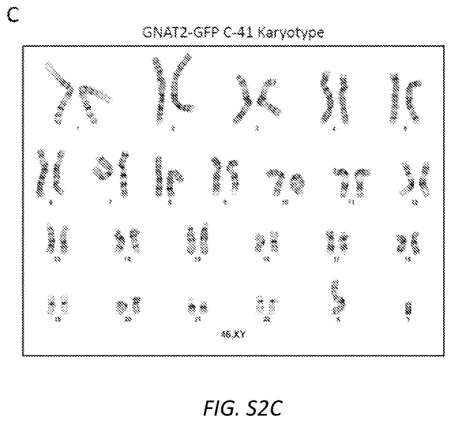

Thus, one embodiment provides a human retinoblastoma organoid model (retinal organoids closely recapitulate human retinal development in vitro (Aparicio, data not shown). Among others, two approaches can be undertaken to model retinoblastoma using retinal organoids. Approach 1) organoids with cone specific, inducible and concerted RB1 KO & EGFP expression. Approach 2) chimeric organoids derived from constitute RB-null cone reporter ESCs/iPSCs plus WT ESCs/iPSCs (FIG. 5). Approach 1) is further described in FIG. 6. And FIG. 7. FIG. 8 provides further details for Approach 1 and 2. FIG. 9 provides details with regards to Approach 2). FIG. 10 details the strategy to generate chimeric RO containing RB-1 null cones, which was used to generate GNAT2-EGFP RB1 KO and characterize the same.

### Example 3

Provided herein are composition and methods of using of GNAT2 knock-in for cone-specific expression of other genes (either instead of EGFP or in addition to EGFP if targeting the two GNAT2 alleles; FIG. 11A). Thus, any gene of interest can be used (i.e., any coding or noncoding cDNA) where constitutive expression in cones is desired. This also includes genes that enable inducible cone-specific expression.

One example includes to insert/knock-in the gene ER^{T2}CreER^{T2} which encodes a tamoxifen-inducible recombinase (Matsuda T, Cepko CL. Controlled expression of transgenes introduced by in vivo electroporation. Proc Natl Acad Sci USA 2007; 104(3):1027-32. DOI: 10.1073/pnas.0610155104), as shown together with EGFP in the other GNAT2 allele to maintain cone specific EGFP expression (FIG. 11A). This can be paired with additional genome editing that respond to the recombinase such as:
flanking a sequence with loxP sites (‘floxing’ (FIG. 11C)), so ER^{T2}CreER^{T2} is expressed, and tamoxifen activated, and the exon deleted solely in cones;
inserting into a locus of interest (e.g., the AAVS-1 safe harbor site) an exogenous promoter-driven Lox-Stop-Stop-Lox preceeding:
   any gene (i.e., cDNA) of interest (GOI), optionally followed by a T2A-Fluorescent protein coding sequence,
   a dCas9^{KRAB} cDNA optionally followed by a T2A-Fluorescent protein coding sequence and with ectopic expression of a sgRNA (used for CRISPRi), and/or
   a dCas9^{VPR} cDNA optionally followed by a T2A-Fluorescent protein coding sequence and with ectopic expression of a sgRNA (used for CRISPRa).

In the above examples (all are in FIG. 11B), tamoxifen treatment induces either the GOI cDNA, dCas9^{KRAB}, or dCas9^{VPR}, solely in cones.

### BIBLIOGRAPHY

Achberger K., et al. (2019). Merging organoid and organ-on-a-chip technology to generate complex multi-layer tissue models in a human retina-on-a-chip platform. ELife, 8, 1-26. 10.7554/eLife.46188
Aparicio J. G., et al. (2023). Optic nerver hypoplasia iPSC derived retinal ganglion cells exhibit differential gene expression with potential relevance to disease etiology. Submitted.
Aparicio J., et al. (2017). Retinal Organoids: An Emerging Technology for Retinal Disease Research and Therapy. 117-138. 10.1007/978-3-319-49479-1_10
Bell C. M., et al. (2020). Human Organoids for the Study of Retinal Development and Disease. Annual Review of Vision Science, 6, 91-114. 10.1146/annurev-vision-121219-081855
Browne A. W., et al. (2017). Structural and functional characterization of human stem-cell-derived retinal organoids by live imaging. Investigative Ophthalmology and Visual Science, 58(9), 3311-3318. 10.1167/iovs. 16-20796
Decembrini S., et al. (2020). Hydrogel-based milliwell arrays for standardized and scalable retinal organoid cultures. Scientific Reports, 10(1), 1-10. 10.1038/s41598-020-67012-7
Gagliardi G., et al. (2018). Characterization and Transplantation of CD73-Positive Photoreceptors Isolated from Human iPSC-Derived Retinal Organoids. Stem Cell Reports, 11(3), 665-680. 10.1016/j.stemcr.2018.07.005
Gasparini S. J., et al. (2019). Transplantation of photoreceptors into the degenerative retina: Current state and future perspectives. Progress in Retinal and Eye Research, 69 (October 2018), 1-37. 10.1016/j.preteyeres.2018.11.001
Gasparini S. J., et al. (2022). Transplanted human cones incorporate into the retina and function in a murine cone degeneration model. Journal of Clinical Investigation, 132(12). 10.1172/JCI154619
Hemshekhar M., et al. (2016). Emerging roles of hyaluronic acid bioscaffolds in tissue engineering and regenerative medicine. International Journal of Biological Macromolecules, 86, 917-928. 10.1016/j.ijbiomac.2016.02.032
Hoshino A., et al. (2017). Molecular Anatomy of the Developing Human Retina. Developmental Cell, 43(6), 763-779.e4. 10.1016/j.devcel.2017.10.029
Kaewkhaw R., et al. (2015). Transcriptome Dynamics of Developing Photoreceptors in Three-Dimensional Retina Cultures Recapitulates Temporal Sequence of Human Cone and Rod Differentiation Revealing Cell Surface Markers and Gene Networks. Stem Cells, 33(12), 3504-3518. 10.1002/stem.2122
Kelley M. W., et al. (1994). Retinoic acid promotes differentiation of photoreceptors in vitro. Development, 120(8), 2091-2102. 10.1242/dev.120.8.2091
Kuwahara A., et al. (2015). Generation of a ciliary margin-like stem cell niche from self-organizing human retinal tissue. Nature Communications, 6 (May 2014). 10.1038/ncomms7286
Lam P. T., et al. (2020). Generation of a retina reporter hiPSC line to label progenitor, ganglion, and photoreceptor cell types. Translational Vision Science and Technology, 9(3), 5-7. 10.1167/tvst.9.3.21
Lu Y., et al. (2020). Single-Cell Analysis of Human Retina Identifies Evolutionarily Conserved and Species-Specific Mechanisms Controlling Development. Developmental Cell, 53(4), 473-491.e9. 10.1016/j.devcel.2020.04.009
Morris T. A., & Fong S. L. (1993). Characterization of the gene encoding human cone transducin α-subunit (gnat2). In Genomics (Vol. 17, Issue 2, pp. 442-448). 10.1006/geno.1993.1345
Morris T. A., et al. (1997). Localization of upstream silencer elements involved in the expression of cone transducin α-subunit (GNAT2). Investigative Ophthalmology and Visual Science, 38(1), 196-206.
Mustafi D., et al. (2009). Structure of cone photoreceptors. Progress in Retinal and Eye Research, 28(4), 289-302. 10.1016/j.preteyeres.2009.05.003
Nazlamova L., et al. (2022). Generation of a Cone Photoreceptor-specific GNGT2 Reporter Line in Human Pluripotent Stem Cells. Stem Cells (Dayton, Ohio), 40(2), 190-203. 10.1093/stmcls/sxab015
Ong O. C., et al. (1997). Gene Structure and Chromosome Localization of the G g c Subunit of Human Cone G-Protein (GNGT2). 109(44), 101-109.
Phillips M. J., et al. (2018). Generation of a rod-specific NRL reporter line in human pluripotent stem cells. Scientific Reports, 8(1), 1-11. 10.1038/s41598-018-20813-3
Ran F. A., et al. (2013). Genome engineering using the CRISPR-Cas9 system. Nature Protocols, 8(11), 2281-2308. 10.1038/nprot.2013.143
Rashidi H., et al. (2022). Generation of 3D retinal tissue from human pluripotent stem cells using a directed small molecule-based serum-free microwell platform. Scientific Reports, 12(1), 1-13. 10.1038/s41598-022-10540-1
Razafsky D., et al. (2016). Lamin B1 and lamin B2 are long-lived proteins with distinct functions in retinal development. Molecular Biology of the Cell, 27(12), 1928-1937. 10.1091/mbc.E16-03-0143
Ronning K. E., et al. (2018). Loss of cone function without degeneration in a novel Gnat2 knock-out mouse. Experimental Eye Research, 171 (December 2017), 111-118. 10.1016/j.exer.2018.02.024
Shayler D. W. H., et al. (2023). Single cell transcriptomics reveals early photoreceptor trajectories and a cancer-predisposed cone precursor state. In revision.
Singh H. P., et al. (2018). Developmental stage-specific proliferation and retinoblastoma genesis in RB-deficient human but not mouse cone precursors. Proceedings of the National Academy of Sciences of the United States of America, 115(40), E9391-E9400. 10.1073/pnas. 1808903115
Sluch V. M., et al. (2017). Enhanced Stem Cell Differentiation and Immunopurification of Genome Engineered Human Retinal Ganglion Cells. Stem Cells Translational Medicine, 6(11), 1972-1986. 10.1002/sctm.17-0059
Tram N. K., & Swindle-Reilly K. E. (2018). Rheological properties and age-related changes of the human vitreous humor. Frontiers in Bioengineering and Biotechnology, 6(DEC), 1-12. 10.3389/fbioe.2018.00199
Wahlin K. J., et al. (2021). CRISPR Generated SIX6 and POU4F2 Reporters Allow Identification of Brain and Optic Transcriptional Differences in Human PSC-Derived Organoids. Frontiers in Cell and Developmental Biology, 9 (November). 10.3389/fcell.2021.764725
Welby E., et al. (2017). Isolation and Comparative Transcriptome Analysis of Human Fetal and iPSC-Derived Cone Photoreceptor Cells. Stem Cell Reports, 9(6), 1898-1915. 10.1016/j.stemcr.2017.10.018
Xu X. L., et al. (2014). Rb suppresses human cone-precursor-derived retinoblastoma tumours. Nature, 514(7522), 385-388. 10.1038/nature13813
Zhong X., et al. (2014). Generation of three-dimensional retinal tissue with functional photoreceptors from human iPSCs. Nature Communications, 5 (May). 10.1038/ncomms5047

All embodiments of any aspect of the disclosure can be used in combination, unless the context clearly dictates otherwise.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present disclosure.

1. A pluripotent stem cell line with a genetic modification that allows expression of either a) a protein and/or or b) a tamoxifen inducible Cre recombinase under control of an endogenous GNAT2 promoter.

2. A pluripotent stem cell derived, in vitro generated, retinal cell line, retinal tissue or retinal organoid (ROs) comprising either a) a protein and/or or b) a tamoxifen inducible Cre recombinase under control of an endogenous GNAT2 promoter.

3. A retinal organoid comprising a population of human pluripotent derived photoreceptor (PR) cells comprising either a) a protein and/or or b) a tamoxifen induced Cre recombinase under control of an endogenous GNAT2 promoter.

4. The cell line of claim 1, wherein the pluripotent stem cell is a human embryonic stem cell (hESC) or a human induced pluripotent stem cell (hiPSC).

5. The cell line of claim 1, wherein the protein is a marker protein.

6. The cell of claim 5, wherein the marker protein is a fluorescent protein.

7. The cell of claim 6, wherein the fluorescent protein is enhanced green fluorescent protein (EGFP).

8. A method of producing a retinal cell line comprising culturing the modified pluripotent stem cell line of claim 1 to form a retinal cell line.

9. The method of claim 8, wherein the retinal cell line is further cultured to form retinal tissue and/or retinal organoids.

10. The method of claim 9, wherein the protein is expressed in immature and mature cone cells in retinal tissue and/or retinal organoids.

11. The method of claim 9, wherein there is minimal disruption of normal cone development.

12. A method for obtaining a retinal tissue or a retinal organoid comprising culturing the modified pluripotent cell line of claim 1 under conditions which allow the cells to differentiate into retinal tissue and/or retinal organoid.

13. The ROs of claim 2, wherein the ROs are embedded in a hydrogel.

14. The ROs of claim 13, wherein the hydrogel comprises hyaluronic acid (HA) and gelatin.

15. The ROs of claim 14, wherein the HA is thiolated-HA.

16. The ROs of claim 13, wherein hydrogel comprises thiol-modified hyaluronan, a thiol-reactive crosslinker (e.g., polyethylene glycol diacrylate), and thiol-modified denatured collagen.

17. The method of claim 8, further comprising episodic live imaging and assessment of cone morphological changes, inner segment development and mitochondria localization.

18. The method of claim 13, wherein the embedded ROs are embedded and optionally imaged for more than 6 weeks without impaired photoreceptor development attributable to the hydrogel.

19. A method of testing for an effective therapeutic treatment for a retinal disease/disorder comprising:
a. producing a retinal organoid comprising the method of claim 9, wherein the retinal organoid optionally has a genetic mutation or other impairment,
b. administering a candidate therapeutic treatment to the retinal organoid of a); and
c. determining the effect of the candidate therapeutic treatment on the retinal organoid of b).

20. The method of claim 19, wherein the candidate therapeutic effect on the organoid is monitored overtime with episodic live imaging and optionally therapeutic is administered more than one time.

21. The method of claim 19, wherein the retinoblastoma 1 gene (RB1) gene is knocked out.

22. The method of claim 19, wherein the candidate therapeutic treatment comprises a protein, a virus, a RNA molecule, a DNA molecule, a gene therapy, a small molecule, a gene editor, a base editor, an RNA editor, a small molecule targeting DNA/RNA, a cell therapy, a genome or base editing technology or a nanoparticle.


SEQUENCE LISTING (45 sequences)

SEQ ID NO: 1 - DNA, 20 bp, Homo sapiens
  source 1..20: mol_type = genomic DNA
aagacggcaaatatgggaag

SEQ ID NO: 2 - DNA, 20 bp, Homo sapiens
  source 1..20: mol_type = genomic DNA
cagatggtaaatatgggaag

SEQ ID NO: 3 - DNA, 19 bp, Homo sapiens
  source 1..19: mol_type = genomic DNA
cagaggtaaatatgggaag

SEQ ID NO: 4 - DNA, 20 bp, Homo sapiens
  source 1..20: mol_type = genomic DNA
aacatggtaaatataggaag

SEQ ID NO: 5 - DNA, 20 bp, Homo sapiens
  source 1..20: mol_type = genomic DNA
accacgtgaaatatgggaag

SEQ ID NO: 6 - DNA, 18 bp, Homo sapiens
  source 1..18: mol_type = genomic DNA
tagaaggtatatgggaag

SEQ ID NO: 7 - RNA, 20 nt, synthetic construct
  source 1..20: mol_type = other RNA
aagacggcaaatatgggaag

SEQ ID NO: 8 - RNA, 20 nt, synthetic construct
  source 1..20: mol_type = other RNA
aagacggcaaatatgggaag

SEQ ID NO: 9 - DNA, 25 bp, synthetic construct
  source 1..25: mol_type = other DNA
caccgaagacggcaaatatgggaag

SEQ ID NO: 10 - DNA, 25 bp, synthetic construct
  source 1..25: mol_type = other DNA
aaaccttcccatatttgccgtcttc

SEQ ID NO: 11 - DNA, 42 bp, synthetic construct
  source 1..42: mol_type = other DNA
gaacatttgagacacgtggcttaagacctgcaggcatgcaag

SEQ ID NO: 12 - DNA, 42 bp, synthetic construct
  source 1..42: mol_type = other DNA
ggtgtatggtgaagagaatggggactctagaggatccccggg

SEQ ID NO: 13 - DNA, 33 bp, synthetic construct
  source 1..33: mol_type = other DNA
aagacggcaaatatggtgagcaagggcgaggag

SEQ ID NO: 14 - DNA, 33 bp, synthetic construct
  source 1..33: mol_type = other DNA
actggctccacttccaggcccgggattctcctc

SEQ ID NO: 15 - DNA, 24 bp, synthetic construct
  source 1..24: mol_type = other DNA
cccattctcttcaccatacaccct

SEQ ID NO: 16 - DNA, 24 bp, synthetic construct
  source 1..24: mol_type = other DNA
atttgccgtcttgtcagctttttc

SEQ ID NO: 17 - DNA, 20 bp, synthetic construct
  source 1..20: mol_type = other DNA
ggaagtggagccagtgctga

SEQ ID NO: 18 - DNA, 24 bp, synthetic construct
  source 1..24: mol_type = other DNA
agccacgtgtctcaaatgttcttg

SEQ ID NO: 19 - DNA, 20 bp, synthetic construct
  source 1..20: mol_type = other DNA
gctggctttagtgggccaaa

SEQ ID NO: 20 - DNA, 20 bp, synthetic construct
  source 1..20: mol_type = other DNA
cttgtggccgtttacgtcgc

SEQ ID NO: 21 - DNA, 20 bp, synthetic construct
  source 1..20: mol_type = other DNA
tacagcctgctctctcacct

SEQ ID NO: 22 - DNA, 20 bp, synthetic construct
  source 1..20: mol_type = other DNA
aggtagaacccaccaaccct

SEQ ID NO: 23 - DNA, 25 bp, synthetic construct
  source 1..25: mol_type = other DNA
gcctctgatcttagtttactagtct

SEQ ID NO: 24 - DNA, 20 bp, synthetic construct
  source 1..20: mol_type = other DNA
actgacaaacagggaagcca

SEQ ID NO: 25 - DNA, 20 bp, synthetic construct
  source 1..20: mol_type = other DNA
tcccaaccatagcacttggc

SEQ ID NO: 26 - DNA, 20 bp, synthetic construct
  source 1..20: mol_type = other DNA
tcccaaccatagcacttggc

SEQ ID NO: 27 - DNA, 20 bp, synthetic construct
  source 1..20: mol_type = other DNA
agtttgcttttgcccagcat

SEQ ID NO: 28 - DNA, 20 bp, synthetic construct
  source 1..20: mol_type = other DNA
attaaggcaggcgccagatg

SEQ ID NO: 29 - DNA, 20 bp, synthetic construct
  source 1..20: mol_type = other DNA
ggaagcagtgagaaaggcca

SEQ ID NO: 30 - DNA, 20 bp, synthetic construct
  source 1..20: mol_type = other DNA
aaggaggtttatggcgcctc

SEQ ID NO: 31 - DNA, 20 bp, synthetic construct
  source 1..20: mol_type = other DNA
atgagctagatggtgcctga

SEQ ID NO: 32 - DNA, 22 bp, synthetic construct
  source 1..22: mol_type = other DNA
aacagaatgcaatgaaggctct

SEQ ID NO: 33 - DNA, 16526 bp, Homo sapiens
  source 1..16526: mol_type = genomic DNA
cctttacacagtgcctggcacgaagtgagggcccagtgaaaggtagctgttatcattgtcccaggattcattttaagcagaagtcctgcccctgcaatctcacttggtcgtctgttttcttccggccggcgtaggtaggaggctgtgccctgcaattctggctccccttcccacatttctgtatttggtggttaagagctttaactctagccagaatgtctgggttcaaatccagatttcacctacttcctgactgatactgggcaagttacctaacttctctgtgcttctatttcgtctgtaaaatgagtgtaagagtacctgctttatatggtggttaagataaatgagttaatataagttaactgcttagaactgtgcctggcctgtagttagtactcagtattcatgtattaccattgctttagccttggccttggttctctattgacactcacaggttgattcacttgcagccgttctggggtctatccaacaagcatccccactgccaaggggtagataatccgacctaaagggaacttgtggagggaagtccctaagacttcactgaatataattaaatgcattagcaggaattaatctcttccctattttccctcttgtgggaggcctttgccccccacacccagttgttctaatgctgtttgtctggctcctcccagttcttaccactcccacagcagagatccttcccaaactctgcctccctcttctctccccacgctggattctggaccctgaacccttttctccacctgcatctggtgccacttccattgatccctggctactgttccctgaacaaacttaaaaaaaacttggcctcttcacctcacacagaatccataaaactaaaaagagaacagagataaccaggtttacctaatcaaaatagcaaagactaaaaaacaaaaaaccaaaaaataaacgctaatgcaggttggcaagggtatagttagtgagatgggagacacacacacacgtacatgtgcacaaacacattactggtggccttgaacactgatataatctttctgaaagccatctggtaatatagaatcaaaattttcaaaaaggttcttattatttgacccagtaatcaactttctggaagttgtttttctaaaaagagctgccagaaaaatatgtatgcaaaaagttgttcagcaccacaatgtgtattgcatagaaaagttagatactttaaaggtcgaacaataggggaatgtttatgtaatattcataccatgaaatacagccattaaaaattgttcttggagaaatgtttaatgatgtggaaatgtttacaatataaagttaggtaaaagaatggatgcaaaacactatacacagtatggtcctacttatgtgatatacatgggtagaaaagaaacacaccaacaagtttaccaatgattatctgtagtgatggaattatgggttatatttattttctttatatttatttgcattttagaaattataatacatacccataacttttataatcaaggggatagaaatcaacaaaaatctgtaaaaatctgtaattgcctccacagtccccccaacccagccttcccggggtcttaaccccagacttaaactattctgtttacatttatggcccactgaattggtcttggggcgcttagggattttgaggctgatggactctcaagaatgaataaaatgaatctcaaaaatcaagcaccatgtaactacataaatgtcatgtatcttctgataagatgcaaatgttgacatagttttaaagatagtatcttgcattgggccaggtctcttgagcatcctgcttaatgcagacctacctacctcagcagccaataggtggcaatccttgccctccacccagcccaggcaaaccagttcattagtccctttatgctagtcccctagtcccatctgcaacacacccttcactctacaaccctcccctatttccaggtcatctggtgtattgttgctgctaggaagtcggccctgattatagttaggcatcaatccctccacccactccataccagcaagaatctcagtgaggtttcattgtctacgcagaacagagcttgggaaataaaggagtgagtcatcctatggcctgggcttttttcacactgaggccttaacattcattgcctttgagcctctgagatattccctagctccaaacacagcttcctcccctcctagcctgttccccacatggctccttaactggccctcccggaatcccagctccctaaagatggtgcactcctgtttctctgaagaagtgcttttctttgcttctggacttcagctcacttgtgatgtccgcctccctagctaggccagaacaatcttcacagatactaaacttgggagagaatatctgcagctccaaaaaggtccttggccaaggcaatttcccaaaccataccttatgcctaggcccttctagaaacaagctgttcagctctggtttctcagcttctcccagcagccagctccctctccccctcacctcagtcacccagcattgtgtttatagcaccatacagctgtttccatctatacttgtttataagaccccagtggattttagtcaaaccccttgccattgaaggcgcttcactggctggctccaatttccctttcttgtcttcatcttcacctcccaaccctccactccatgcacctttcactccagtccagtcacatcaaagtcccagtcttctctgagcacaccttgtactttgtgcctctacctcttttttcatgctctttcctttccccaaaatgcgtatctcccacttccactgttattctgcttctctgtggaaagtctattcatttcacaatcacccttttctttcttccgctgtagaactttgtcagtctgacagtcagatgtgtgtttgattgcctttcctaccaggggtgaatgctaggtcttaaacagctctgaatttcccagcactactgcagtatccctttaactcatttgtttttctgtgaatttcccccacctctgcctatggactggaaggtcctgaaggtagttcctcatccttcatgtcctcccactccatctgggacaagtctttacccacaattcacttaagacaaggcttaccacggatgtcagacagatcccttaactcaggaaacatgcccaaatcactaagaaacagtgatttacttttcattcaaaatgataatggttataaagggctgtgaatcaaaacacctgagctctggcttggcttctgataccaacttgcttattaatgtgaggatcacttttctgtacttcagttcactcacctccaaaatggaactagcactacataccttatttacttcatcaggaaactgttcgtaaatgtgatcataaagtgggacagccacaggaaaggcacaggaactgacgatagaggcagaggcaggaggtcacagacatcaggatgatgtatgcacagtaatggatctgtactctcaggccaggcgagggctagcagttccctctctcagcttagcagtgagaaaggggaacttccatccagtaagttagggaacaaagcagaagcagcacagaagtttgcagaggtagtagcagaaaaaggaacaagcccactttccctgtgtgtgaaacaacttagtggttcctttaacaagggctgctgttatagagttcatgcatttgtccatgctttctctctctggctacttatgtggaagtaatagaaagaaggatcaccggtttgtctgtttactaggccatataactttgtttttttgtttttgaaacagagtctcactctgtcacccaggttggagtgcagtggcgcaatctcggctcactgcaacctccacctcccgggttcaagcgattctcatgcctctatctcccaagtagctgggtgcatgccaccatgctgggctttttcttttcttttcttttcttttttttttttttttctgagacggaatcttgctctgttgcccaggctggagtgcagtgcctcccgggttcaagtgattctcctgcctcagcctcccaagtagctgggattacaggtgcctgccaccatgcctggctaatttttgtattttttttaagtagagacagggtttcaccatgttgaccaggctgtctcgaactcctgacctcagatgatctgcccattttggcctcccaaagtgttggaattacaggcgtgagccaccatgcccggcctaatttttgtatttttagtggatacgggatttcaccatgttggccaggaactcctgacctcaggtcatccacccacctcagcctcccaaagtgctgggattacaggcatgcgccactatgcccagctgactttaaatgagttacttaactctcaagtctcagttaatctagtctgtaaattggggacaatagcagttattgtgataattaactggtatgagccacttacattgttcagcagttattaattataataactataacataataattattaatagttccaggcagccccttgaacagtgtagccttcagcatggctctggcctcagtgtgagttaccagtattgtgagtacgctggagcagggctagcagtggggtataattactgaaaccttataaccaagattccaagatagtcacaatttcaaagagactctcctgtttttccttaagtcaataaggacttgccaatttgattgggaaaacatggctaccatggaaagcacctcagcagctgcatatttctccatcagcccctacagtcactgaaaggtggtgcgaaggaggaagataattagctatggctcagggtacctgataggcggggagacctagattctactcctgaccttcccagtcccaactggccactgctgcacaagtccagctctaaaatgaaagagcaaattacatccttcgacctcagtagttctccaccttggctgtacactagaatcagctggggacgtttttaaatccttgtgcctggactgcactccagaccaattaaatcagtattttttaaaaagctctccaggtaaatgccactgtacactaaaagttgagaactgtttcaggccagggtttttcaaacttcacagatgtgataactacctgtattagcttaaaatattttctctggctctggagtttgatgctaacataccatttaaaaacatacactattttaacatctcggaaagcagggtaatcttctagtcagtggcacctaagacttgaggaaacacagacaccagcaacccaggtgattcttaggattaggtaagtttagcggaaaacaggtccctagggcagtacttcccaatctttaatgtctttagagtcacctgagatcttgttaaactgcggattctgaatcagaagttctggattaaagcctgagattctgcatgtctaatgagcttccgggtgatctgatgttgctggctcttgatccacattttgggcaacaaaattctaaaacatctccacctgaggaggctccgccagcaacactcatttaagtaaactgaaataattggcatagaggagtacaacctgtggagtcctaaatgtctgtgaggcagttgctagactgcaccctcatttttcccccaggaaagggcagctgggatgagagccagaaggagagagagctgccccaacctttgagaagccagagtctggagtccaatttcccaaagaagcagagttttttgtgtgaggcagcacaaaccccacactgaataccagcaaagttcatttatgaagtgaagttgggactcagctggctttagtgggccaaagggaagcaaccccattctcttcaccatacacccttttcctgcatttattcattcaccaaaccttttgatccacaaataaactacaagttctgagaggttcaaagatgagtcagatttggtccgttgagctccagggggagaaatgcagtgagggaaaagatttgtaaaacgacgtacaatatgaagtgataagtgctaataacagaagtacaaagagaggggccaatgcaaaggaacaatgctagtagcttcctggagcagcagccactgcgggagctgaaacattcctaatcttcccaaggaagggcaccacccaaaacaaatttcctggccaggaccagcctatggtaaacgagtatgctttgataccctgaagcccttgagatcaagaccttataatctggaggctcaacataaggaatgctttctacatatgtgccagtaatctctagctctatgatgcaaataaatctaaggaagcaagagactttcaggggatgaaccccttaaaggatggaagtagtcgtgcatcctatccttccgtcagaacccagcagatcatttccctagttatagaaacatttgagtctttaccccttgccatattgacaaagctcttaattggcttgacctatcacattgctagatataaaggctacaatccctagactaagaagtaggtctccagttgaagtagggagtctcagtcaatgtaggcagagtacaagaccctacagcctgctctctcacctgccatcgtacagaccagcttttaggggagccaagttgggatactcaatcccaacttttttccttctcttccatctcacatacaggaaaccttacgagagaggattaggggcctgaaaaagctgacaagacggcaaatatgggaagtggagccagtgctgaggacaaagaactggccaagaggtccaaggagctagaaaagaagctgcaggaggatgctgataaggaagccaagactgtcaagctgctactgctgggtgagtgagatgggaagatgagccagagaaggcaggggtccttcctactttcctgaagggttggtgggttctacctcaccccatgggaaggaagggtggcaggtcatttttcctctcctctacacagtctggctaggggatcaggagatctagagctgagttaatatggggcctaaacagccaccccaaggggatccagaatgccaaggctattccagagtttttctactcttgagcgaggaatagtgcaagggccaccatgaacccacccatttccaaatctaaccaaacctaacacatccttttggcttcaaggaccctggacttgcagactgtacccaatctgcagagaactctaagccaagaaatcagaagagaacaggaccttccctcaccaacaggctcacaagtcccaccatacagtcagtgccaacagtaccagagatagtcccactggtttttgccaagtatagtggttccttcttgctttcagtaaaaactttggaagtagggggctctgaggaaggaggaatggtgtctttatgtacagcagtcccttcctggctctctattcaatagctgcctgcaaagctcctgccagatggaaggttcatcaacttgatgagctcctaagcagatcactggtctgtgctgagaaaataaaagcacctcaatttgtcagggaaattgatcacagctgtaaataaaaccaagacaagaacatttgagacacgtggcttaggaaaacaaaccactggtaccacagaagtagggtagctggagcaggtagggtctacgtagcagaagattagatgcctgagctgggtttccaagcccccataagggatctgggagctgacgcactaggctaaggcaccttcttttcccccagctgatctgtggcacagtcgtaaggacacactaaaggagcatatctttgtaagctggaccagactctaaggagcccaggaggttacgcagggggaaacagagatggtggggccactgagagatcttttaagcctaagcagatttcttctacattcaggataagctgcttagagggaacaagcacaagcgaaataggaggagttcgaggcattagggtagtataaactcagtactagaaggtatgagtttttgatggagagagcagagtgtgaatgaggacattaggacacattagtcaataaagggaacccacttagccccatccaagaccaggttgagcaacatggtgaaaacctgcctctacggaggggcggggggaaatggccggatgcagtggcgcacgcctgtaatcccagcgctttgggaggccaagatgggtggatctacctgaggtcaggagtttgagaccagcctggccaacatagtgaaaccccatttctactaaaaatacaaaaattagtcaggcatggtggcatgcgcctgtagagtcccagctactcaggaggctgaggcagaagaattgcttaaaacccaggaggcagaggttgcagtgagccaagatagcaccactgcactccagcctgggagacagagcaagactgtctcaaaaaaaaaaaaaaaaatgtagtcgggcatgatgtcgcacacctggaatcccaactactcaggagactgaggtgggaggatcacttgagcctgggaagtcaaggctgcagtaagccgagattgtgccattacactccagcctgggctacaaacctgagaccctgtctcaaaaaaaaaaaagggaaccagcaaggtgatgaaaatatagttattttggtgaaatgatccagctctctccaatcctaccccaagatcactctcctgagtcaaaaataggcagagaggagaaatgttaaaggactccccctgaatctgttagtggttttcaacaggaagattttgactgccagtggtcatttggaaatgtctggagacacttttggttgtcataactagggaagtgggatgctattggcctctagtgggtagaggccaggaatgctgctgaacatcctaccatgcatgagacagcccaaactaaatagtactgaagttaagaaactgctctaaatccaggttgaatggcctgagctcaagcctgccagaaattgagggcagcagtcatccctatgtattctcccctaacaagacccccaagcaagcagtggctctgacttctcccaggccatctcctggaaggctgaggagaactggtggaaatcgaaagcataagcatttttccttccaggtgctggggagtcaggaaagagcaccatcgtcaaacagatgaagtgagtagaaacaaagccccaaaagacaagatagggtgaagaagtcagtacagccagtggaggtattcaaagtgaaaggctctttagcctcaaggagcccaggtataaaggatctgattccaatgcccctcatctgtaccccttgtccctcaccctccactttgagaaagcagtagcaacagagagataggattcttatgatcctttaaatacccccaaattcctaatcccttaggtctggttactcaggtccagctaaagacagagtgtctgccccttgcaggatcattcaccaggatggctattcaccagaagaatgcctggagttcaaggctatcatctatggaaatgtgctgcagtccatcctggctatcatccgggccatgaccacactgggcatcgattatgctgaaccaagctgtgcggtatgtgattactattatgtggttaagggtggaagcagaaaggctagcaagaagaaacataccagaggccaacaaactatatggaaagatgggtaagaaaaatagtaactaaaatcccacctgctgggtgggatctcactgctaggtgtactgtgtataccatctcaaggccctttacccatctacgcagtaagaccttaaggtagacggtacctttctaattagcctcattttacacatggaaaaactgaagttcagagaagttaattaactgcccaaggtcatgtagataaatagcagagactgcatttgcattgggctgcttgactacaaagctgaatatttttcataatacaccacaattatgcagtcctggagaagtaagcaacatccacccttatttttactgggacagggtcttcctctcttacccaggctggaatgcagtggtgtgatcatggctcactgcaatctcaccctcccaggcttaagtgattctcccacctcagccccagtaggtgggaccacaggtgtgcaccaccacacccagctattttttaaaatttttttgtagagaaggggtcttgccatcttgtctaggctggtgtcaaactcctggccttaagcaatcctcctgcctcagactccgaaagtgttgagattacaggtgtgagccaccgtgcctgtcctcaagccctactttataacacatatttacaaaataaagctgctcacagcttcctctgcttttttactttaccatttaaattgctacccttgggttcctggccatggaacttttgtaagtgaaatccctacttctagtactggactttttctagctttcaatcgctgaagaacaaaggtgatgggtccctgtcttctatcttgtctatttcatggtcaatctggcctttacattttcagtctctctgcagcagaggacccacgttttgtggcggtaaactttcagactaaggatggaagccagaaactccagagaggaaatacctgggacccctgccccgctttagacacaccccagggctgggtatagtgccaccctctgcatctggggagcatactcaaaaattcaacagtatgttttcttacatagaatcttcactggatactgcttccatcttaggtcttcgtagtaatgtaaaatgatttccattcagcagtattcccagaagcctcctggaaagctattactcctgtgaagttcttaaccaggtttctgcattacaggatgacgggcgacagctcaacaacctggctgactccattgaggagggaaccatgcctcctgagctcgtggaggtcattaggaggttgtggaaggatggtggggtgcaagcctgcttcgagagagctgcagaataccagcttaatgactccgcatcttagtaagactgactggtgagagggtgggttgatgcttaagcaatcttctagccagtcttctctctggttgggagaaacctcacccaacccaaaatttcaggcattgaaagctggagacccagactgaattcagcctgtggatctgttttgttaggcttcagcaatgttttgaatttaatgctatggggagatctgccacagttgtcatgactttctattgctttacactggctcactacagcctacaaggctgaagatgcaaaattcaaaagacgtgttctgcctagtagccctcagccctgttctcttaattgaggtgtacactaagcctctccttccagaaccctaagatagccctgcagttctgtcatacactctcgcaggagtaatgtgattcatgatggctctttcagggcagaattttctatctggtaatctaggatctacttaggccactaagcagaaatgagtctcctacacctagagcatcttatgtcacgatgccaaggcaggggacacaaactacaagggcaaaccaagggaaggtggtcaggaattagtaccatcattgcaaagctgcttccaaaaagctaagggacataatttatcaatcctacctcagacagtatgggatcatagacaagtatgggcttcaaagtcaaacctgagtttatttaacctaaccatctgtctcctaatcttgtaataatgctatcacttactgttcccggagcactcaaagcccagtcactgctaggtgtactgtgtataccatctcaacgccctttacccatctacgcagtaagaccttaaggtagatggtacctttctaattagcctcattttacacatggaaaaactgaagttcagagaagttaattaactgcccaaggtcatgtagataaatagcagagactgcatttgcattgggctgcttgactacaaagctgaatatttttcataatacaccacaatttctcttgaccctagccaagtatttaactcttctgaatttctccatctgtaaaatggggatatgaaacttgatggcttaggagtttaaacgagaatctgaacattaaaatgcctggcatacaataggcacttgtgttacttgtacttccctcctcctctcagtcatctcagttcacatctgccttcctcttgccttttttttttttggagacggagtctcgctttgtcacccaggctggagtgcagtggcgcaatctcggctcactgccagctttgcctcccgggttcacgccattctcctgcctcagcctcccaagtagctggggatactggcgcccgccaccatgcccagctaattttttgtatttttagtagagatggggtttcaccgtgttagccaggatggtttcgatctcctgaccttgtgatccgcccgcctcggcctcccaaagtactgggattataggcgcgagccaccgtgcctggccaccttttttttttttaaaaaaaataactttttcttttttaaggtaactttcaaacatacaaaagtagaaataacataatgaattaccttcaacagttatcaactcatggccaatcttatttctttttacctccattccctactggattattttgaagcaaattccaggtatcatttcatcctaaaaatgtcattatggatctctaaaatattgaccctttttataaaaagaatataatcacacccaaatagttaacaattccttaatagcatcaaatatcaccaagtcggtgttcaaatttccccagttatctcaaatgtttcctccccagtttgattcaggatataaacaaagtccatgtgttgcattggttcatgtctttaaagtctctttcaatttactgcctcttgatggaacttttagcacccactaaatctcattcaaaaatctacaaaagcttgcagtcagggcaaacccaaggaaaacttatctaacttgatttaaatgcgctggaactccagcctcaaatgggcttgtccctcagtccatcccccatttcctctagttaatgacatatgaaggtttgaaggaaagaatcctaaatttaacaatatggcttctagtcctgatttctatcaccaaattagctgtgtgactttgggtaagataccttttacgtctcttagcctcgtctgtggaaaatttgtcattgatgctaatcacttttctctagctacctgaaccaattagaacgaattacagaccctgagtacctccctagtgagcaagatgtgctccgatccagagtcaaaaccacgggcatcattgaaaccaagttttccgtcaaagacttgaatttcaggtaagtgcatggttccctagggcatctcggatacacgtgtggaatcctgaaaaggggcacaggcaggcttcttggcctttggtgaagcctaatctaaattactgttctgcttctcccccttttcctctcccacttcacggggtgaccatcaaaggcatataggtcgtatgttgggcatacctatgaaaatagctgcttcttccctcaggatgtttgatgtgggagggcagagatccgagagaaagaagtggatccactgcttcgagggagtcacctgcatcattttctgtgcagccctcagtgcctatgatatggtgctggtggaagatgacgaagtggtgagtggcctttgcatcaagcagctttggtagaacaagttctccccatgaccctttctctaagccttgtgtcactctactgccccaacttaggtaatttcagtctagcagccctccagcagaccaatcaatgtctcatgcaaataattctaaaaaacaacttcttctgcaggttctagattagcattttagagctccaaatttactgacagtgagcttggtctcaaattagacatctaagtatcacttggacatcacaaagctcataagaggaattgagtgcaaagagataagggaccatcaactaggcaaagcaaaggagttacacttagtactctcccaaattgcctaaggaaggagatgaaaatgacagaacagagaaaataacatatgatatgaatcttcattgcaacataatagaagggttgagctagtaaccccacttaggaggctaaaaatgtactgtccgtaggagtttaaggagagactggcagaccagctttctctcatgccaattaaattggcagctggaagactacccaagagtggttctctttagcctgtagaattctgtaggacaggagttctataggacaagtgttagagcccagccagtttctgaatttgggaaaggttagaggtgagaaaaacgttaatttcacccaagcatctgctttctgaatttgggaaaagttagaggcgtgaaaaacgttaatttcacccaagcatctgctcacaaatggaggtccacaccttgctgatgacctctgaatttgggaaaggttagaggcgtgaaaaatgttaatttcacccaagcatctgcttacaaatggaggtccacagcttgctgatgaaagggatactcctatcccttgccacagcttgttctcttcccttccctttggtagttttaacttcacattagagcactctgaatatcgtctaatcaaaatgtcttacagagctattcacttcccatctttaagcctaaagattacagtctatgagactttccatctttaaccctaaagtctatgagtctatgaggtttattaaagtctatgagacattaataaaacaagtctatgagaccttaaagggttgtacaggagtattatggggaaaaagccacaaatgggattgttcttgctttattagataagtagactgaacgcaagtcagctgatagtatacttcaaaaccctaaagacctgctccctaaaagcaagctgggctggggcaatgggcagcctctgcagatatgcagcccgacttctcgctaagtagcaatcagagaaggaaatgagagagcagaaatgcttgtggtatggcactgggaaattctctaactctcaccatgtggcagcaggaccaaagtagcccaaactgagatctgggaccccatgaaagaagcctatcaaaatcatcctggagatgcatatgggcacatgctaacttgggcctgtttcaacccattatcagcactacttataaaatgtcaagttctcagttgcatcctggctgctaaagatctgcataacacattatagacctatatgccagccactatcatggacaatatacatacacaatctcatttagctttcattgtaaccctataagataggaaaacagactcagaaaaagctcaataattttccacaagtcacacagctattagaaagatagggaactagaatgatcccacatctctctggctttactctggtacattgagataagctgttctctgtcttgctttttacatttggagcactggtctctccaaggggaacaagagcaggaagtaggtagatattctataagccaaatctgatatttccaatggtgtttcctcttactagaatcgtatgcatgagtctttgcatctgttcaacagcatatgtaaccacaaattctttgcggctacttccattgtcctctttctcaacaagaaggacctctttgaggaaaaaatcaagaaagtccatctcagcatttgttttccagagtatgatggtaagtgtcaggggctggaaataataataatgccttttagtagagactggcaattgtctcattttttaggccaagatgacacaaaggaacttaagggagaaccttgggcacagttacagggtttaaattcagatactctggaatacagcaggcattagatgcaggagagccactgacttcatatgatacctactgaaaaccaaaggtggaaagacacctctcctcaatttcttttcaactaaagtgagaaacactggagtgcaatagagaatcttccctccaaaaataggcccccaactgctgttgtctaataacatttcaaggatcaagtcaatcacctaaagtgagtcagcaactaacaagggttcatttattctactttttcactatttttctggaaaaccaggtaacaactcctatgatgatgcggggaattacataaagagccagttccttgacctcaatatgcgaaaagatgtcaaagaaatctacagtcacatgacctgtgctacagatacacagaatgtcaaatttgtgtttgatgcagttacagatattatcatcaaagaaaacctcaaggactgcggcctcttctaatcctcaccattcctcaggtataagttctataaacaggcttggaatctgggtaattaaaaacagaaaattatagtcaatataccatgacatgaagaatgaatccattctttggagatggagtatacatgactgcaactgtatttcatacgttcttttcaaagtgggatagctattgcagcttaaagagcacaggccagtagttagaagaccccccaggttccagtactggttttccaacttaatacaaaactgtgaatacttta

SEQ ID NO: 34 - DNA, 100 bp, Homo sapiens
  source 1..100: mol_type = genomic DNA
accttacgagagaggattaggggcctgaaaaagctgacaagacggcaaatatggtgagcaagggcgaggagctgttcaccggggtggtgcccatcctggt

SEQ ID NO: 35 - DNA, 119 bp, Homo sapiens
  source 1..119: mol_type = genomic DNA
caagggatctggagcaacaaacttctcactactcaacaagcaggtgacgtggaggagaatcccgggcctggaagtggagccagtgctgaggacaaagaactggccaagaggtccaagga

SEQ ID NO: 36 - DNA, 100 bp, Homo sapiens
  source 1..100: mol_type = genomic DNA
attatgtatggtttatgaagaggaaaatatagtctgacagcagatggtaaatatgggaagtaggaagtaattggggtcgtgtgaataatgagttaaaaat

SEQ ID NO: 37 - DNA, 100 bp, Homo sapiens; identical to SEQ ID NO: 36
  source 1..100: mol_type = genomic DNA

SEQ ID NO: 38 - DNA, 100 bp, Homo sapiens
  source 1..100: mol_type = genomic DNA
aactacaggggacaaagaggtcacccacttaaaaaacccacttcccatatttacctctgaggcaatatattaaatttttcataggaaaactttaaaaatt

SEQ ID NO: 39 - DNA, 100 bp, Homo sapiens; identical to SEQ ID NO: 38
  source 1..100: mol_type = genomic DNA

SEQ ID NO: 40 - DNA, 100 bp, Homo sapiens
  source 1..100: mol_type = genomic DNA
aaggaagtaaacacaatggagtgatgaggggaaaggggaaaacatggtaaatataggaagtagcgctaacagcaggagcagtacttctaaatccaaatgg

SEQ ID NO: 41 - DNA, 100 bp, Homo sapiens; identical to SEQ ID NO: 40
  source 1..100: mol_type = genomic DNA

SEQ ID NO: 42 - DNA, 100 bp, Homo sapiens
  source 1..100: mol_type = genomic DNA
ttatggcgcctcataattcgtgggacaggggaggagggtcaccacgtgaaatatgggaagtgggtgatgctgagggctgggggaggggacaggaggtgag

SEQ ID NO: 43 - DNA, 100 bp, Homo sapiens; identical to SEQ ID NO: 42
  source 1..100: mol_type = genomic DNA

SEQ ID NO: 44 - DNA, 100 bp, Homo sapiens
  source 1..100: mol_type = genomic DNA
ccccccatatatatattctttttctgaagaggaactttattagaaggtatatatgggaagagggatggctcagaattggaatatatgctaagttttataa

SEQ ID NO: 45 - DNA, 100 bp, Homo sapiens; identical to SEQ ID NO: 44
  source 1..100: mol_type = genomic DNA